(19) United States
(12) Patent Application Publication
(10) Pub. No.: US 2024/0150847 A1
(43) Pub. Date: May 9, 2024

(54) Highly Sensitive EGFRvIII Detection

(71) Applicant: The General Hospital Corporation, Boston, MA (US)

(72) Inventors: Leonora Balaj, Lincoln, MA (US); Bob S. Carter, Belmont, MA (US); Syeda Maheen Batool, Boston, MA (US)

(21) Appl. No.: 18/503,936

(22) Filed: Nov. 7, 2023

Related U.S. Application Data
(60) Provisional application No. 63/545,170, filed on Oct. 21, 2023.
(60) Provisional application No. 63/382,557, filed on Nov. 7, 2022.

(51) Int. Cl. C12Q 1/6886; C12Q 1/6851

(52) U.S. Cl. CPC C12Q 1/6886; C12Q 1/6851; C12Q 2600/158

(57) ABSTRACT

Provided herein are methods for the detection of EGFRvIII mutation in patient samples, e.g., in tumor tissue, cerebrospinal fluid, and plasma samples, using an optimized ddPCR assay. This methods can be used, e.g., for diagnosis, monitoring tumor progression, and assessing response to therapy.

### CLAIM OF PRIORITY

This application claims the benefit of U.S. Provisional Application Ser. No. 63/382,557, filed on Nov. 7, 2022, and 63/545,170, filed on Oct. 21, 2023. The entire contents of the foregoing are incorporated herein by reference.

### FEDERALLY SPONSORED RESEARCH OR DEVELOPMENT

This invention was made with Government support under Grant Nos. CA069246, CA239078, CA237500, and CA230697 awarded by the National Institutes of Health. The Government has certain rights in the invention.

### TECHNICAL FIELD

Provided herein are methods for the detection of EGFRvIII mutation in patient samples, e.g., in tumor tissue, cerebrospinal fluid, and plasma samples, using an optimized ddPCR assay. These methods can be used, e.g., for diagnosis, monitoring tumor progression, and assessing response to therapy.

### BACKGROUND

Glioblastoma (GBM) is the most common and aggressive primary brain tumor with a global incidence of 0.5 to 5 per 100,000 persons^{1,2,3} and a median survival time of 15 months from the time of diagnosis^{4,5}. Despite increasing advances in therapeutics, prognosis remains poor with high rates of treatment failure and relapse.

### SUMMARY

Provided herein are methods that include detecting EGFRvIII in a sample. In some embodiments, the methods comprise: providing a sample comprising a biofluid, e.g., serum or cerebrospinal fluid, from a subject, preferably a human subject; isolating extracellular vesicles comprising RNA from the sample; reverse transcribing the RNA in the presence of 7-deaza-deoxyguanosine 5′-triphosphate (7 dG) (optionally with a final concentration of about 0.1-0.5 mM, e.g., about 0.25 mM) to create cDNA; using polymerase chain reaction (PCR), amplifying any epidermal growth factor receptor v III (EGFRvIII) sequences present in the cDNA to generate EGFRvIII amplicons, preferably wherein the amplicons are between 50 and 100 bp in length; detecting the presence of EGFRvIII amplicons. In some embodiments, the methods include a step of pre-amplifying the EGFRvIII sequences. In some embodiments, the sample comprises tumor tissue (e.g., from a biopsy).

In some embodiments, the sample is from a subject who is known or suspected to have cancer. In some embodiments, the cancer is glioma, breast cancer, non-small cell lung cancer, head & neck squamous cell carcinoma, colorectal cancer, or prostate cancer. In some embodiments, the glioma is glioblastoma.

In some embodiments, the amplification and/or the preamplification is performed using Forward primer (5′-GGCTCTGGAGGAAAAGAAAGGTAATT-3′; SEQ ID NO:8) and/or Reverse primer (5′-CCGTCTTCCTCCATCTCATAGC-3′; SEQ ID NO:9).

In some embodiments, the amplification and/or the pre-amplification is performed using digital droplet PCR, preferably in the presence of betaine, e.g., in the presence of about 0.25-1.0 mM betaine, e.g., about 0.5 mM betaine.

In some embodiments, the amplification is performed in the presence of an EGFRvIII mutant probe comprising a detectable moiety, and detecting the presence of EGFRvIII amplicons comprises detecting amplicons comprising the detectable moiety. In some embodiments, the probe comprises 5′-TGACAGATCACGGCTC-3′; SEQ ID NO:10.

Also provided herein are methods of detecting an EGFRvIII positive cancer in a human subject who is known or suspected to have a cancer. The methods comprise providing a sample comprising a serum or cerebrospinal fluid from the subject; isolating extracellular vesicles comprising RNA from the sample; reverse transcribing the RNA in the presence of 7-deaza-deoxyguanosine 5′-triphosphate (7 dG) (optionally with a final concentration of about 0.1-0.5 mM, e.g., about 0.25 mM) to create cDNA; using polymerase chain reaction (PCR), amplifying any epidermal growth factor receptor v III (EGFRvIII) sequences present in the cDNA to generate EGFRvIII amplicons, preferably wherein the amplicons are between 50 and 100 bp in length; detecting the presence of EGFRvIII amplicons. The presence of EGFRvIII amplicons indicates that the cancer is EGFRvIII positive. In some embodiments, the sample comprises tumor tissue (e.g., from a biopsy).

In some embodiments, the methods further comprise administering to the subject a treatment for EGFRvIII positive cancer, optionally EGFR inhibitors such as erlotinib, gefitinib, afatinib, and dacomitinib; immunotherapy such as rindopepimut (CellDex), ICT-107, HSPPC-96 Vaccine (Vitespen), EGFRvIII peptide vaccine (Ad.p53-DC), and anti-EGFRvIII CAR-T cell therapy; erb-B inhibitors such as CI-033 (Pfizer); and VEGF/VEGFR inhibitors such as bevacizumab and HKI-272 (Cediranib), as well as combinations thereof with surgical resection and radiation therapy.

In some embodiments, the methods include a step of pre-amplifying the EGFRvIII sequences.

In some embodiments, the sample is from a subject who is known or suspected to have cancer. In some embodiments, the cancer is glioma, breast cancer, non-small cell lung cancer, head & neck squamous cell carcinoma, colorectal cancer, or prostate cancer. In some embodiments, the glioma is glioblastoma.

In some embodiments, the amplification and/or the preamplification is performed using Forward primer (5′-GGCTCTGGAGGAAAAGAAAGGTAATT-3′; SEQ ID NO:8) and/or Reverse primer (5′-CCGTCTTCCTCCATCTCATAGC-3′; SEQ ID NO:9).

In some embodiments, the amplification and/or the pre-amplification is performed using digital droplet PCR, preferably in the presence of betaine, e.g., in the presence of 0.25-1.0 mM betaine, e.g., about 0.5 mM betaine.

In some embodiments, the amplification is performed in the presence of an EGFRvIII mutant probe comprising a detectable moiety, and detecting the presence of EGFRvIII amplicons comprises detecting amplicons comprising the detectable moiety. In some embodiments, the probe comprises 5′-TGACAGATCACGGCTC-3′; SEQ ID NO:10.

As used herein, “about” means±10% unless otherwise indicated.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIGS. 1A-E. RNA folding and secondary structures occurring in the EGFR transcript variant III. (a) Diagram depicting nucleotide sequence of the target amplicon (5′-GGCTCTGGAGGAAAAGAAAGGTAATTATGTGGTGACAGATCACGGCTCGT GCGTCCGAGCCTGTGGGGCCGACAGCTATGAGATGGAGGAAGACGG-′3) in the deleted variant of the EGFR transcript (EGFRvIII) (top, SEQ ID NO:1) and wild type EGFR (bottom, TATGTCCTCATTGCCCTCAACACAGTGGAGCGAATTCCTTTGGAAAACCTG CAGATCATCAG, SEQ ID NO:2). Forward and reverse primers as well as probe sequence specific to each amplicon are illustrated. (b) A bar graph comparing the relative abundance (percentage) of different nitrogenous bases (G+C, A+T) in the target amplicons (light grey=EGFRvIII, dark grey=EGFRwt). (c) Graphical representation of GC content distribution across the length of the target amplicon in both sequences (EGFRvIII, dark grey=96 bp, EGFR wt, light grey=62 bp). Calculation of GC content (percentage) is performed using the formula, (G+C)/(A+T+G+C)*100 with the window size set to 20 bases. (d) Output of secondary structure analysis using Mfold. The individual base pair interactions in EGFRvIII and EGFRwt target amplicon are illustrated using circle graphs. Each arc represents a unique base pair interaction; shown are SEQ ID NOs. 3 and 4. (e) Effect of 7 dG on the secondary structures and the copy number of EGFRvIII (top) and EGFRwt (bottom) in tumor tissue as shown in dd PCR 1D plots.

FIGS. 2A-Q. EGFR transcript variant vIII droplet digital PCR (ddPCR) optimization. (a, b, c) ddPCR 1D plots with mutant channel amplitude (top) and GAPDH channel amplitude (bottom), run in parallel with (right) and without (left). Ethanol precipitation of EGFRvIII cDNA from three different Reverse Transcription protocols; (a) standard, (b) standard+1 μl 7-deaza-GTP) (c) (standard+2 μl). (d, e, f) Two-tailed t-test results depicting the difference in Copies/20 μl from ddPCR using EGFRvIII cDNA treated with and without ethanol precipitation from different RT protocols; (d) standard, (e) standard+1 μl 7-deaza-GTP) (f) (standard+2 μl). (g) Two-tailed t-test results comparing the statistically significant difference in Copies/20 μl in EGFRvIII cDNA from standard and (standard+1 μl 7-deaza-dGTP) RT protocols, both untreated with Ethanol precipitation. (h) Two-tailed t-test results comparing the statistically significant difference in Copies/20 μl in EGFRvIII cDNA from (standard+2μl 7-deaza-GTP) and (standard+1 μl 7-deaza-dGTP) RT protocols, both untreated with Ethanol precipitation. (j) Two-tailed t test results comparing the statistically significant difference in Copies/20 μl in EGFRvIII cDNA from standard and (standard+1 μl 7-deaza-dGTP) RT protocols, treated with Ethanol precipitation. (k) Two-tailed t test results comparing the statistically significant difference in Copies/20 μl in EGFRvIII cDNA from (standard+2 μl 7-deaza-GTP) and (standard+1 μl 7-deaza-dGTP) RT protocols, treated with Ethanol precipitation. (i) QQ plot obtained from one way ANOVA results depicting statistically significant difference in average Copies/20 μl in EGFRvIII cDNA across different conditions overall. (1) Two-tailed t test results depicting statistically significant difference in Copies/20 μl in EGFRvIII cDNA (standard+1 μl 7-deaza-dGTP, RT protocol) purified using different cleanup protocols. (m) ddPCR 1D plots demonstrating change in separation of mutant events from the baseline at different annealing/extension temperatures (low vs high). (n) Quantitative difference in Copies/20 μl of mutant events across different annealing/extension temperatures. (o) Violin plots demonstrating statistically significant difference in Copies/20 μl with the addition of 0.5 mM Betaine, 0.5 mM EDTA, 0.5 mM (Betaine+EDTA) versus no addition of ddPCR additive (control). (p) ddPCR 2D plots (top row) and 1D plots depicting cluster density, tightness, and separation of mutant events and GAPDH events at different concentrations of Betaine versus no Betaine addition to ddPCR. (q) Varying numbers of EGFRvIII synthetic RNA were spiked into the reverse transcription reaction. The resulting cDNA was then amplified using the optimized ddPCR (top) and qPCR (bottom) cycling conditions. For ddPCR copies per 20 μl are plotted against EGFRvIII copies spike-in (top). Limit of Detection (LOD, dashed line) is plotted, defined as 3 standard deviations over average copies per 20 μl obtained when only a small concentration of EGFRvIII was run along with blanks. Limit of Blank (LOB, dashed line) is plotted, defined as the apparent highest copy number expected to be found when replicates of a blank sample containing no EGFRvIII (or small concentration of EGFRvIII) are tested. For qPCR Cycle threshold (Ct) is plotted against EGFRvIII copies spike-in (bottom).

FIGS. 3A-I. Higher Prevalence of EGFRvIII in tumor tissue using 7-deaza-dGTP (7 dG). (a) Experimental workflow for detecting EGFRvIII mutation in tumor tissue. (b) Heatmap of EGFRvIII Ct values obtained by qPCR in tumor tissue RNA from cohort 2, in parallel, with and without 7-deaza-dGTP. (c) Two-tailed t test depicting the statistically significant difference in the Ct value obtained, with and without 7-deaza-dGTP. (d) EGFRvIII prevalence in cohort 2. (e) Pearson's correlation of normalized EGFRwt (x-axis) and normalized EGFRvIII (y-axis) (reference gene, GAPDH). (f) Heatmap of EGFRvIII Ct values obtained by qPCR in tumor tissue RNA from cohort 3, in parallel, with and without 7-deaza-dGTP. (g) Two-tailed t test depicting the statistically significant difference in the Ct value obtained, with and without 7-deaza-dGTP. (h) EGFRvIII prevalence in cohort 3. (i) Pearson's correlation of normalized EGFRwt (x-axis) and normalized EGFRvIII (y-axis) (reference gene, GAPDH). (p≤0.00001,****), (p≤0.0047,**), (p≤0.0493,*)

FIGS. 4A-B. Detection of EGFRvIII mutation in plasma samples of patient cohort (n=3). (a) Schematic depicting experimental workflow, including isolation of plasma, extraction platform, reverse transcription, purification and subsequent ddPCR amplification. (b) Contingency tables were constructed from data obtained, and sensitivity and specificity were calculated and graphed above. Overall sensitivity and specificity across all three cohorts are also reported.

FIGS. 5A-D. Detection of EGFRvIII mutation using optimized plasma-based assay in varying clinical outcomes. (a) EGFRvIII mutation (copies/20 μl) in serial plasma samples obtained from four glioma patients are plotted against time (months postoperatively). Patient cases represent different clinical outcomes: (a), (b) recurrent disease, (c) stable disease, and (d) inconclusive MRI i.e. inability to clearly delineate pseudo progression from true tumor progression. T1-Weighted, contrast enhanced MRI images are provided for different clinical timepoints. Surgical procedures are indicated using a square (GTR=gross total resection). Disease progression (tumor recurrence) is indicated in (a) and (b) using a shaded background. Treatment courses for each patient are outlined.

FIGS. 6A-C: Secondary Structure prediction in target amplicon of EGFRvIII and EGFR wt using Mfold algorithm. (a) Four distinct secondary structures in target amplicon (96 bp) of EGFRvIII with minimum free energy (ΔG) in the range of (−32.87 to −37.70) kcal/mol; shown is SEQ ID NO. 5, GCCCGGCGAGUCGGGCUCUGGAGGAAAAGAAAGGUAAUUAUGUGGUGACA GAUCACGGCUCGUGCGUCCGAGCCUGUGGGGCCGACAGCUAUGAGAUGGA GGAAGACGGCGUCCGCAAGU). (b) Depiction of nine potential pseudoknots in target amplicon of EGFRvIII as predicted by algorithm; (c) Three possible secondary structures predicted in EGFR wt target amplicon (62 bp, SEQ ID NO:6, TATGTCCTCATTGCCCTCAACACAGTGGAGCGAATTCCTTTGGAAAACCTGCA GATCATCAG) with ΔG in the range of (−4.33 to −7.20) kcal/mol.

FIGS. 7A-C: Analysis of nucleotide sequence (467 bp) unique to EGFRvIII at exon1:exon8 junction site (a) Nucleotide sequence (AGACGTCCGGGCAGCCCCCGGCGCAGCGCGGCCGCAGCAGCCTCCGCCC CCCGCACGGTGTGAGCGCCCGACGCGGCCGAGGCGGCCGGAGTCCCGAG CTAGCCCCGGCGGCCGCCGCCGCCCAGACCGGACGACAGGCCACCTCGTC GGCGTCCGCCCGAGTCCCCGCCTCGCCGCCAACGCCACAACCACCGCGCA CGGCCCCCTGACTCCGTCCAGTATTGATCGGGAGAGCCGGAGCGAGCTCTT CGGGGAGCAGCGATGCGACCCTCCGGGACGGCCGGGGCAGCGCTCCTGGC GCTGCTGGCTGCGCTCTGCCCGGCGAGTCGGGCTCTGGAGGAAAAGAAAG///////////GTAATTATGTGGTGACAGATCACGGCTCGTGCGTCCGAGCCTGTGG GGCCGACAGCTATGAGATGGAGGAAGACGGCGTCCGCAAGTGTAAGAAGT GCGAAGGGCCTTGCCGCAAAG, SEQ ID NO:7) of exons 1:8 fusion where EGFRvIII occurs, highlighting GC, CG and G repeats (double underlined above). (b) Graphical representation of the GC content (percentage) in the nucleotide sequence of exon1:exon8 with the calculation based on window size of 30 bases. (c) Circle graph obtained from secondary structure prediction analysis illustrating the individual base pair interactions in 20 predicted foldings. Each color represents a distinct base pair interaction. The ΔG indicating the thermal stability of the predicted foldings is −213 kcal/mol.

FIGS. 8A-B: Summary of optimization measures. (a) Amplification plot demonstrating the effect of using oligodT and Random hexamers in different combinations during reverse transcription on resulting Ct value in qPCR. (b) Melt curve plots illustrating the maximum peak and Tm value of the qPCR product in the three different reverse transcription conditions.

FIGS. 9A-H: Summary of qPCR optimization measures. Amplification plot (a) and melt curve plot (b) obtained by testing different concentrations of forward and reverse primers. Amplification plot demonstrating the Cycle threshold (Ct) values obtained at different probe concentrations (c). Annealing temperature gradient (54-60°) C assessment to determine the peak of target product and prevalence of non-specific products as shown on melt curve plots (d). Schematic depicting the three step (e) and two step (f) qPCR cycling conditions. Bar graph (g) demonstrating the lowering of Ct value at four different forward/reverse primer concentrations with the use of three step (open bars) vs two step (grey bars) cycling conditions. Melt curve plots (h) from two step (top panel) and three step (bottom panel) cycling conditions at four different primer concentrations comparing the maximum peak of the target product.

FIGS. 10A-B: Reproducibility and intratumoral heterogeneity. (a) Linear regression (top) and correlation matrix (bottom) between copies/ml and clinical parameters. (b) Detection of EGFRvIII mutant levels in plasma derived EV RNA, in a group of EGFRvIII patients (P10, P16, P20) and controls (Wt3, Wt5, Wt8) at two different time points, T1 and T2. The copies of EGFRvIII are normalized to GAPDH run in the same well. Third column represents EGFRvIII copies (normalized to GAPDH) in longitudinal samples.

FIGS. 11A-F: Assessment of assay efficiency and linearity. EGFRvIII cDNA reverse transcribed from serially diluted tumor tissue RNA was amplified using (a) ddPCR, and (d) qPCR. (a) Linear Regression graph depicting the EGFRvIII copies detected via ddPCR plotted against the RNA input. Regression analysis reported R2=0.99. (b) ddPCR 1D plots demonstrating the mutant signal generated at each RNA concentration. (d) qPCR standard curve demonstrating Cycle threshold (Ct) values plotted against the log RNA input. Linear regression analysis reported R2=0.99. (c) ddPCR 1D plots depicting the mutant signal generated when EGFRvIII synthetic RNA is reverse transcribed into cDNA with (+) and without (−) 7 dG followed by ddPCR amplification. (e) ddPCR 1D plots demonstrating the mutant signal generated when cDNA from serially diluted EGFRvIII synthetic RNA was amplified using ddPCR. C number corresponds to the data point and the EGFRvIII copy number input in each RT condition. (f) ddPCR 2D plots (top row) and 1D plots depicting cluster density, tightness, and separation of mutant events and GAPDH events at different concentrations of EDTA versus no EDTA addition to ddPCR.

FIGS. 12A-B. Detection of EGFRvIII mutation in CSF and plasma derived EV RNA. Liquid biopsy was performed on extracellular vesicles (EV) derived from (A) cerebrospinal fluid (CSF) and (B) peripheral blood from a human patient diagnosed with GBM using the methods described above at several time points throughout two treatment cycles with a targeted immunotherapy.

FIG. 13. Exemplary schematic. An exemplary workflow for a detection method as described herein.

### DETAILED DESCRIPTION

Tissue biopsy is the current gold standard diagnostic tool for GBM. However, in addition to being invasive and not always feasible, tissue biopsy only provides a single time point evaluation of the tumor genomic profile, thereby limiting its utility in exploring the tumor heterogeneity spatially and over time. Therefore, there remains the need for development and clinical implementation of alternative sensitive, non-invasive modalities to facilitate serial sampling and real-time monitoring of evolving genetic and epigenetic alterations. In this regard, liquid biopsy, defined as sampling and analysis of non-solid biological fluids (CSF, plasma), offers new opportunities for GBM molecular characterization and detection of prognostic biomarkers⁶. Tumors including gliomas have been shown to shed biological material into the circulation which in turn reflects the transcriptomic and proteomic profile of cell of origin. Examples of analytes include extracellular vesicles (EVs), circulating tumor cells (CTCs), cell-free DNA (cfDNA), proteins, and metabolites. Tumor derived extracellular vesicles (EVs) are particularly promising for biomarker discovery because they encapsulate and retain tumor specific mRNAs in a stable form in a range of biofluids. In addition, the enclosed cargo can vary in response to diverse stimuli and tumor stage⁷.

Previous work established detection of several key molecular markers in biofluids from patients with glioma, including EGFRvIII mRNA, IDH1 RNA/DNA, TERT promoter, and PTEN deletion^{8,9,10,11}. Epidermal growth factor receptor (EGFR) amplification represents one of the hallmark alterations of GBM and is present in about 57% of primary GBMs and 8% of GBMs arising from recurrent low grade gliomas^{12,13}. Approximately, 30-50% of EGFR-amplified GBMs harbor an in-frame deletion of exons 2-7, forming a truncated receptor with loss of extracellular ligand binding domain¹⁴. This deleted EGFR variant III (EGFRvIII) is capable of constitutive signaling in a ligand independent manner thereby promoting tumorigenesis and overall tumor growth. In contrast to EGFR wild type (EGFRwt), EGFRvIII is only expressed in tumor cells, thereby making it a viable therapeutic and diagnostic target in precision oncology¹⁵. Clinical testing and validation of EGFRvIII from biofluids has been hampered by several challenges, namely, limited understanding of structural biology of EGFRvIII, use of suboptimal methods of tumor specific EV isolation, low sensitivity of employed PCR technologies, and overall low sensitivity and/or specificity of reported methods.

The present inventors observed that the EGFRvIII mRNA transcript contains several four guanine (4G) repeat sequences, especially close to the junction site (exon1:exon8), which is also the target region of interest. In other sequences, 4G sequences have been shown to play a role in formation of G-quadruplexes as well as other 3-D secondary structures^{16,17} that are known to play an inhibitory role in reverse transcription and PCR amplification^{18,19} Addition of PCR additives may destabilize secondary structures, as previously reported for the amplification of the highly GC rich TERT promoter region²⁰.

To the best of the present inventors' knowledge, RNA secondary structures in EGFRvIII mRNA have not been previously discussed in the literature. Here we sought to establish a sensitive assay for the detection of the EGFRvIII mutation in EV-derived RNA, optionally using plasma-based droplet digital PCR (ddPCR). As described herein, several variables underwent optimization, including minimizing the adverse effects of secondary structures at the reverse transcription and PCR steps, by using different PCR additives. Furthermore, to improve amplification efficiency and specificity we optimized thermocycling conditions, temperature gradients, and purification protocols. Using the overall optimized protocol, the prevalence of EGFRvIII mRNA was determined in tumor tissue from 37 tumor tissue samples. Finally, the new assay was tested in 54 plasma samples that included tissue confirmed EGFRvIII (n=30), EGFRwt (n=10), and age matched healthy controls (n=14).

This study demonstrates methods for the detection of EGFRvIII mutation in tumor tissue and glioma patients' plasma and CSF samples using an optimized assay. The assay had an overall sensitivity of 72.8% and a specificity of 97.7% for detecting EGFRvIII in plasma compared to tumor tissue analysis. The assay revealed a higher prevalence of EGFRvIII in EGFR amplified tumor tissues (˜80%) as compared to what was previously reported in the literature. Using this platform, we showed clinical usefulness of this technique in patients with glioma with several applications: diagnosis, monitoring tumor progression, and assessing response to therapy.

Development of a robust and sensitive assay for detecting EGFRvIII mutation posed substantial technical and structural challenges. Secondary structures in the EGFRvIII RNA sequence have not been previously described in literature. There are two main types of algorithms commonly used for RNA secondary structure prediction: Deterministic Dynamic programming algorithm, and Minimum free energy algorithm. The former is based mainly on the measurement of frequency of base pair interactions with the assumption that the formed base pairs are discontinuous, thereby having low accuracy²³. Based on the Nussinov algorithm, Zuker proposed a minimum free energy algorithm, a more robust prediction tool based on the principle of interaction of secondary structures with free energy and neighboring base pairs^{21,22}. Hence, this was also used for predicting secondary structures in EGFRvIII RNA. Structural analysis of the predicted 3D foldings using crystallography and other sophisticated modalities is beyond the scope of this study. However, it is worth mentioning that thermodynamically stable secondary structures were also found to be highly prevalent close to the junction site (fusion of exon 1 and exon 8), a nucleotide sequence unique to EGFRvIII mutation. This presented a challenge in primer design and selection due to primer dimers and Tₘ mismatch. This could potentially lead to non-specific products thereby limiting the specificity of the assay. In addition, secondary structures have a well-documented role in reducing the efficiency of polymerase activity and overall efficacy of PCR amplification. Hence, we sought to overcome these challenges through reverse transcription enhancement via 7-dG and development of an amplification protocol that effectively utilized betaine as a potent reaction buffer. 7-dG, a modified nucleotide, is capable of selectively blocking Hoogsten bond formation, without interfering with normal Watson-Crick base pair interactions²⁴. A role for betaine in improving amplification of GC rich regions in TERT promoter mutation has also been reported previously where it was used in conjunction with EDTA²⁵. However, while we did see an improvement in separation with EDTA, there was a simultaneous lower mutant fluorescent signal. Hence, we finally used betaine alone at optimized concentration of 0.5 mM. Based on the present findings of the prevalence of secondary structures in EGFRvIII, future studies are necessary to further elucidate EGFRvIII structural biology. Additional optimization measures included amplicon length and ddPCR optimization. Short amplicon length, optimized for fragmented plasma nucleic acid²⁶, combined with betaine, known to act as an iso-stabilizing agent, significantly improved the binding of oligonucleotides to the target region^{27,28}.

Using the optimized assay, we were also able to detect EGFRvIII mutation with a higher sensitivity in tumor tissue. In this study, overall tumor tissue EGFRvIII prevalence measured in two cohorts was 81%. The estimated EGFRvIII tissue expression in existing literature is about 24%-67% of GBMs²⁹. Our study population included patients with confirmed EGFR amplified status. As per literature, approximately 46% of GBM lesions are EGFR amplified, with EGFRvIII present in about 50% of such cases^{30,31,32}. However, the present results showed a significantly higher prevalence of EGFRvIII in EGFR amplified tumor tissue. This not only highlights the sensitivity of this assay but also raises an important question on the true prevalence of EGFRvIII in GBM patients and our findings suggest that it is higher than previously thought. Since EGFRvIII expression is tumor cell specific, this makes it a potentially useful therapeutic target^{33,34,35}, which has led to a growing interest in developing anti-EGFR/EGFRvIII therapeutics including antibodies (cetuximab, panitumumab, nimotuzumab), vaccines, chimeric antigen receptor (CAR) T cells and RNA based treatments¹³. Hence, with more accurate clinical classification of patients in clinical settings and their subsequent enrollment in relevant targeted clinical trials can potentially lead to better clinical outcomes and lower rate of treatment resistance, offering new opportunities in precision oncology.

We note that a sensitivity of 72.77% and a specificity of 97.67% in the plasma of 30 patients with EGFRvIII confirmed tumors approximates findings previously achieved using CSF as a biofluid for EGFRvIII detection with clinically relevant sensitivity¹⁰. However, lumbar puncture (LP), an invasive procedure, is required to obtain CSF. In this regard, plasma is preferable with the ability to do serial sampling. The present methods have multiple clinical applications: 1) the potential of circumventing tissue biopsy in patients who are not favorable candidates for surgery or have a tumor in an inaccessible location; 2) monitoring disease burden and treatment response; 3) patient stratification and selection of potential candidates for clinical trials and/or targeted therapeutics; and 4) companion surveillance tool to aid in clinical decision making and differentiate pseudoprogression and/or treatment induced effects from true disease. In a small number of patients with longitudinal samples changes in EGFRvIII detection at different time points was concordant with different clinical outcomes: stable disease, tumor progression, and treatment effectiveness, including a patient where increased plasma EGFRvIII was detected at a follow-up time point was observed while there was no clear radiological delineation between radiation necrosis vs tumor progression. Another patient with effective treatment response and stable MRI findings showed no detectable EGFRvIII in follow-up plasma and CSF analysis. The correlation of EGFRvIII positivity in plasma with tumor progression may have important implications as previous studies targeting EGFRvIII via rindopepimut vaccine have reported the ability of tumor to escape through selection and preferential proliferation of cells that are EGFRvIII negative. In addition, the one “false positive” sample detected in our blinded cohort had a recurrent contrast enhancing lesion with confirmed EGFR amplification. Tissue analysis did not reveal EGFRvIII mutation, however, in plasma 2 mutant events were detected in one well and 1 mutant event was detected in the second well (2/4 wells), which highlights the sensitivity of the present assay, and potential tumor heterogeneity and the variable EGFRvIII expression in tumoral versus peritumoral areas in tissue³⁶. Thus in some cases liquid biopsy may provide a more sensitive sampling of the entire tumor compared to a spatially limited tissue biopsy.

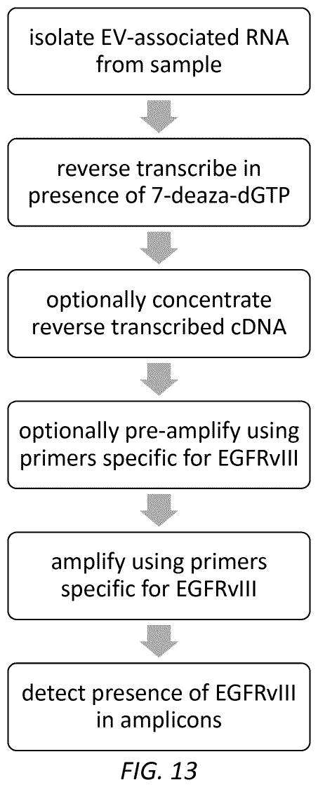

Thus, provided herein are methods for detecting EGFRvIII in a sample from a subject; FIG. 13 provides an exemplary workflow.

As used herein the term “sample”, when referring to the material to be tested for the presence of a biological marker using the methods described herein, includes inter alia tumor tissue (e.g., from a biopsy), and biofluids, e.g., whole blood, plasma, serum, saliva, lymph, urine, or cerebrospinal fluid (CSF), or exosome or exosome-like microvesicles or other extracellular vesicles (collectively referred to herein as EVs; see, e.g., U.S. Pat. No. 8,901,284; Enderle et al., PLoS One. 2015 Aug. 28; 10(8):e0136133; Akers et al., J Neurooncol. 2015 June; 123(2): 205-216) isolated from any of the foregoing. In some embodiments, the sample is serum or CSF. Various methods are well known within the art for the identification and/or isolation and/or purification of a sequence from a sample. For example, nucleic acids contained in the sample can be isolated according to known methods, for example using lytic enzymes, chemical solutions, or isolated by nucleic acid-binding resins following the manufacturer's instructions.

An exemplary workflow is shown in FIG. 13. Preferably, the methods first include isolating EV-associated RNA from the sample, e.g., a sample comprising plasma or CSF. Methods for isolating EV-associated RNA are known in the art and can include ultracentrifugation (Akers et al., J Neurooncol. 2015 June; 123(2): 205-216), size-based filters (e.g., ExoMir, Biooscientific), antibody-based capture (e.g., Immunobeads, HansaBioMed), polymer-based precipitation reagents (e.g., Life Technologies, System Biosciences Inc.), and spin column-based methods; see, e.g., Enderle et al., PLoS One. 2015 Aug. 28; 10(8):e0136133. Commercial kits, e.g., ExoRNeasy Kits for purification of total vesicular RNA (Qiagen), Total Exosome RNA & Protein Isolation Kit (Invitrogen™), or SeraMir Exosome RNA Purification Kit (System Biosciences) can be used.

As shown in FIG. 13, after isolation of EV-associated RNA from the sample, the method uses reverse transcription in the presence of 7-deaza-2′-deoxyguanosine 5′-triphosphate (7 dG or 7-deaza-dGTP) (e.g., stock concentration of 5 mM) to prepare cDNA, optionally with a final concentration of 0.1-0.5 mM, e.g., about 0.25 mM, and optionally betaine, with a final concentration of 0.25 mM-1.0 mM, e.g., about 0.5 mM. Without wishing to be bound by theory, it is believed that the presence of 7 dG compensated for the secondary structure present in EGFRvIII, and allowed for more complete reverse transcription. The reverse transcription reaction preferable includes both oligo dT (e.g., Oligo d(T)₂₀) and random hexamers, optionally in a 2:1 to 1:2 ratio, optionally 1:1 molar ratio.

The cDNA can optionally be concentrated, e.g., using methods known in the art such as precipitation with ethanol or isopropanol, or extraction using an organic solvent such as butanol. In preferred embodiments, ethanol precipitation with sodium acetate and glycogen is used, e.g., as described herein.

The cDNA can also be pre-amplified and then amplified, e.g., using a polymerase chain reaction (PCR) method, e.g., droplet digital PCR (ddPCR) or digital PCR. In some embodiments, the amplification and optional preamplification is performed using the following primers: Forward mRNA primer (5′-GGCTCTGGAGGAAAAGAAAGGTAATT-3′; SEQ ID NO:8), Reverse mRNA primer (5′-CCGTCTTCCTCCATCTCATAGC-3′; SEQ ID NO:9). Other primers, e.g., as shown herein, can also be used.

The presence and/or level of an EGFRvIII nucleic acid (e.g., gDNA or mRNA) as described herein can be evaluated using methods known in the art. For example, the presence of an EGFRvIII mutation can be assayed, e.g., using PCR, e.g., reverse transcriptase polymerase chain reaction (RT-PCR), quantitative or semi-quantitative real-time RT-PCR, droplet digital PCR (ddPCR), digital PCR, e.g., BEAMing ((Beads, Emulsion, Amplification, Magnetics) Diehl (2006) Nat Methods 3:551-559). DNA or RNA can also be assayed, e.g., using nucleic acid sequencing (e.g., Sanger, pyrosequencing, NextGeneration Sequencing, or Long Read Sequencing). See, e.g., Lehninger Biochemistry (Worth Publishers, Inc., current addition; Sambrook, et al, Molecular Cloning: A Laboratory Manual (3rd Edition, 2001); Bernard (2002) Clin Chem 48(8): 1178-1185; Miranda (2010) Kidney International 78:191-199; Bianchi (2011) EMBO Mol Med 3:495-503; Taylor (2013) Front. Genet. 4:142; Yang (2014) PLOS One 9(11):e110641); Nordstrom (2000) Biotechnol. Appl. Biochem. 31(2):107-112; Ahmadian (2000) Anal Biochem 280:103-110.

In some embodiments, high throughput methods, e.g., protein or gene chips as are known in the art (see, e.g., Ch. 12, Genomics, in Griffiths et al., Eds. Modern genetic Analysis, 1999,W. H. Freeman and Company; Ekins and Chu, Trends in Biotechnology, 1999, 17:217-218; MacBeath and Schreiber, Science 2000, 289(5485):1760-1763; Simpson, Proteins and Proteomics: A Laboratory Manual, Cold Spring Harbor Laboratory Press; 2002; Hardiman, Microarrays Methods and Applications: Nuts & Bolts, DNA Press, 2003), can be used to detect the presence and/or level of an EGFRvIII nucleic acid or protein as described herein. For example, oligonucleotide probes that bind specifically to an EGFRvIII junction sequence can be used.

In some embodiments a technique suitable for the detection of alterations in the structure or sequence of nucleic acids, such as the presence of deletions, amplifications, or substitutions, can be used for the detection of biomarkers of this invention. For example, PCR can be used to detect EGFRvIII, either by comparing amplicon length (wherein the presence of an amplicon that is longer than expected if there was no EGFRvIII present) or by sequencing the amplicons (wherein the EGFRvIII can be directly detected by comparing the amplicon sequence to a reference or control sequence that represents a wild type sequence in the absence of an EGFRvIII event).

Preferably, the presence of amplicons of EGFRvIII can be detected using probes that comprise a polynucleotide sequence that binds to EGFRvIII as described herein, optionally wherein the probes are immobilized on a solid support or surface or on beads. For example, the probes can comprise DNA sequences, RNA sequences, co-polymer sequences of DNA and RNA, DNA and/or RNA analogues, or combinations thereof. The probe sequences can be synthesized, e.g., chemically or enzymatically. In some embodiments, the probe comprises 5′-TGACAGATCACGGCTC-3′; SEQ ID NO:10. Optionally, the probe comprises one or both of a reporter comprising a detectable moiety at the 5′end, e.g., FAM™, JOE™, NED™, ROX™, SYBR® Green I, TAMRA™, TET™, VIC™, and/or a minor groove binder (MGB) and/or a quencher, e.g., a nonfluorescent quencher (NFQ) at the 3′end, optionally MGB and NFQ.

In some embodiments, presence of EGFRvIII is detected as described herein, and the subject has one or more symptoms associated with cancer, e.g., the subject has a tumor (e.g., identified by imaging or palpation), then the subject can be diagnosed with cancer. In some embodiments, the subject has no overt signs or symptoms of cancer, but the presence of EGFRvIII is determined using a method as described herein, then the subject can be identified as having an increased risk of developing cancer or of recurrence of an EGFRvIII+ cancer. In some embodiments, once it has been determined that a person has cancer, or has an increased risk of developing cancer, then a treatment, e.g., as known in the art or as described herein, can be administered. In some embodiments, presence of wt EGFR, and/or absence of EGFRvIII, is detected as described herein, and treatment decisions can be made accordingly.

In some embodiments, once presence of EGFRvIII is detected using a method described herein, the subject can be further evaluated using methods known in the art to confirm a diagnosis of cancer.

Although the present methods are exemplified in GBM, the methods can also be used in patients with other cancers. The term “cancer” includes malignancies of the various organ systems. In some embodiments, the cancer is a carcinoma, adenocarcinoma, or sarcoma. Cancers include those affecting brain, lung, breast, thyroid, lymphoid, gastrointestinal, and genito-urinary tract, colon cancers, renal-cell carcinoma, prostate cancer and/or testicular tumors, non-small cell carcinoma of the lung, cancer of the small intestine and cancer of the esophagus

The term “carcinoma” is art recognized and refers to malignancies of epithelial or endocrine tissues including respiratory system carcinomas, gastrointestinal system carcinomas, genitourinary system carcinomas, testicular carcinomas, breast carcinomas, prostatic carcinomas, endocrine system carcinomas, and melanomas. In some embodiments, the disease is renal carcinoma or melanoma. Exemplary carcinomas include those forming from tissue of the cervix, lung, prostate, breast, head and neck, colon and ovary. The term also includes carcinosarcomas, which include malignant tumors composed of carcinomatous and sarcomatous tissues. An “adenocarcinoma” refers to a carcinoma derived from glandular tissue or in which the tumor cells form recognizable glandular structures, and include malignancies such as most colon cancers, renal-cell carcinoma, prostate cancer and/or testicular tumors, non-small cell carcinoma of the lung, cancer of the small intestine and cancer of the esophagus. The term “sarcoma” is art recognized and refers to malignant tumors of mesenchymal derivation.

For example, the following table shows overall prevalence of EGFRvIII mutation across different systemic cancers.

| Disease | Prevalence (%) | Reference |
|---|---|---|
| Glioblastoma | 25-81 | 37, 38 |
| Breast cancer | 67.8 | 39 |
| Non-Small Cell Lung cancer | 16-42 | 40, 41, 42 |
| Head & Neck Squamous Cell | 4.4-42 | 43, 44 |
| Carcinoma | | |
| Colorectal cancer | 8.1 | 45 |
| Prostate cancer | 6.5 | 45 |

The present methods can be used to determine the presence of EGFRvIII in other cancers as well, since previous methodology may have under-reported the presence of EGFRvIII in other cancers.

In some embodiments, once presence of EGFRvIII is detected as described herein, the subject can be treated, e.g., with a treatment for cancer, e.g., chemotherapy, immunotherapy, radiotherapy, or surgical resection. In some embodiments, a treatment specifically for EGFRvIII+ cancer is used, e.g., EGFR inhibitors such as erlotinib, gefitinib, afatinib, and dacomitinib; immunotherapy such as rindopepimut (CellDex), ICT-107, HSPPC-96 Vaccine (Vitespen), EGFRvIII peptide vaccine (Ad.p53-DC), and anti-EGFRvIII CAR-T cell therapy; erb-B inhibitors such as CI-033 (Pfizer); and VEGF/VEGFR inhibitors such as bevacizumab and HKI-272 (Cediranib), as well as combinations thereof with surgical resection and radiation therapy. The methods can also be used to stratify subjects in a clinical trial, e.g., to associate presence of EGFRvIII with response or non-response to a candidate treatment. In addition, the methods can be used to monitor subjects who are undergoing treatment, to identify subjects as responders or non-responders, and to identify subjects who are having a recurrence of an EGFRvIII+ cancer.

Also provided herein are kits for use in the present methods, e.g., comprising oligonucleotides and reagents for amplification and detection of EGFRvIII, e.g., as described herein. Optionally the kits comprise reagents for reverse transcription that comprise 7-deaza-dGTP; for example, the reagents can comprise oligo dT (e.g., Oligo d(T)₂₀), random hexamers, dNTP mix, deaza-dGTP, buffer, DTT, ribonuclease inhibitor, and reverse transcriptase (e.g., SuperScript™ IV).

### Examples

The invention is further described in the following examples, which do not limit the scope of the invention described in the claims.

Methods

The following materials and methods were used in the Examples below unless otherwise noted.

Tumor Tissue Processing

Tumor tissue was microdissected and suspended in RNAlater (Ambion) or flash-frozen and stored at −80° C.

Patient Plasma Processing.

Whole blood was collected using K2 EDTA tubes with an inert gel barrier (BD Vacutainer Blood Collection Tubes), from pre-operatively placed arterial lines or venipuncture. Within 2 hours of collection, samples were centrifuged at 1,100×g for 10 minutes at 20° C. to separate the plasma from the hematocrit and filtered using 0.8 μm filters. 1 ml aliquots were stored at −80° C. for later downstream analysis. Except for the longitudinal samples, all baseline samples were collected prior to surgical resection.

Cell Lines

The glioma cell line Gli36-EGFRvIII was kindly provided by Xandra O. Breakefield and cultured in DMEM (Invitrogen) containing 10% fetal bovine serum (FBS) (Sigma Aldrich) and 5% penicillin/streptomycin (Sigma Aldrich). Messenger RNA (mRNA) was isolated from cultures at 50-70% confluency. Cells were frequently tested to ensure absence of mycoplasma contamination (Mycoplasma PCR Detection Kit; Applied Biological Materials).

Synthetic RNA

Nucleotide sequence of the EGFRvIII mRNA transcript around the region of interest (120 bp) was purchased as Ultramer RNA oligonucleotide (Integrated DNA Technologies). Amount of Oligo was 68.49 nmol and purified using standard desalting. The quality of the synthetic RNA was not optimal due to the high GC content. The ultramer RNA oligo was then resuspended in nuclease free water to make upto 100 μM. Initial quantification using NanoDrop™ Spectrophotometer (ThermoFisherScientific) demonstrated RNA concentration of 4,430 ng/μl. Aliquots were prepared and stored at −80.0° C. Accurate qualitative and quantitative analysis of the RNA was performed using Agilent RNA 6000 pico kit run on Agilent Technologies 2100 Bioanalyzer (Waldbronn, Germany). For this purpose, the sample was diluted 2000-fold (based on Nanodrop measurement). Electropherograms obtained from Bioanalyzer revealed two peaks and we manually selected for the concentration (area under the curve) of the peak of interest based on the RNA size (120 bp). The resulting nanograms were then converted to EGFRvIII copy number using the standard copy number formula for single stranded RNA as shown below.

$$number of copies ( molecules ) = \frac{Xng * 6.0221 \times {10}^{23} molecules / mole}{{( N * 660 g / mole )}^{†} * 1 \times {10}^{9} ng / g}$$

Where: X=amount of amplicon (ng)
N=length of ssRNA amplicon
660 g/mole=average mass of 1 bp ssRNA

Study Population

The study population (n=54, n=30 EGFRvIII positive, n=10 EGFR wild type, n=14 age matched healthy controls) included 53 patients aged 18 years or older who underwent surgery at the Massachusetts General Hospital (MGH) for biopsy or resection of a primary brain lesion. Clinical EGFRvIII status was established with either immunohistochemistry (IHC) or by the MGH SNaPShot Panel. Additional inclusion criteria for the study population included histopathological confirmation of disease. Healthy control patients with a history of oncologic, neurologic, or ongoing infectious conditions were excluded from the study. All samples were collected under an IRB approved protocol with informed patient consent. Patient demographics are provided in Table 1.

RNA Isolation from Cell Lines and Tumor Tissue

RNA was isolated from cell lines using the RNeasy Kit (Qiagen, Germantown, MD, USA) per manufacturer's recommendations and eluted in 20 μL of nuclease free water (Qiagen). Frozen tissue was thawed and lysed in 1-2 mL of ice-cold TriZol Reagent (ThermoFisher). Lysate was homogenized by passing through a 20-gauge RNAse-free needle 10 times. Total RNA was then extracted as per the manufacturer's protocol and eluted in 20 μL of nuclease free water (Invitrogen). All RNA samples (cell lines, tumor tissue) were assessed for purity with the NanoDrop One spectrophotometer (ThermoFisher, Waltham, MA, USA,). Agilent RNA 6000 pico kit was used with Agilent Technologies 2100 Bioanalyzer (Waldbronn, Germany) to determine the concentration and RIN (RNA Integrity Number) value of the samples. Isolated RNA was stored at −80° C. prior to downstream analysis.

EV RNA Extraction from Plasma and CSF Samples

In order to isolate the analyte of interest, 2 ml plasma or CSF was obtained from each clinical sample and thawed on ice. Extracellular vesicle RNA (EV RNA) was isolated using the ExoRNeasy Maxi Kit (Qiagen), per manufacturer's recommendations. EV RNA was eluted in 17 μL of nuclease free water (Qiagen) and assessed for concentration and purity with the NanoDrop One spectrophotometer (ThermoFisher Scientific).

Optimized Reverse Transcription

Messenger RNA (mRNA) isolated from tumor tissue and cell lines, and EV RNA isolated from plasma samples, was reverse transcribed into cDNA using the SuperScript™ IV First-Strand Synthesis System (ThermoFisher Scientific). The protocol was optimized to maximize cDNA yield. In the first part, 1.0 μl 50 μM Oligo d(T)₂₀, 1.0 μl 50 μM Random Hexamers, 1.5 μl 10 mM dNTP mix and 1.0 μl 7-deaza-dGTP (NewEngland BioLabs) at a final concentration of 0.25 mM were combined with template RNA (1.0 ng) in a 0.2 ml PCR reaction tube. Nuclease free water was then added to reach a final reaction volume of 13 μl. The RNA-primer mix was then incubated at 65° C. for 5 minutes, immediately followed by incubation on ice for 1 min. In the second part, Reverse Transcription (RT) reaction mix was prepared by combining 4.0 μl 5× SSIV Buffer, 1.0 μl 100 mM DTT, 1.0 μl Ribonuclease Inhibitor, and 1.0 μl SuperScrip™ IV Reverse Transcriptase (200 U/μl) in a reaction tube to a final volume of 7 μl. The contents were briefly vortexed and centrifuged. The RT reaction mix was then added to annealed RNA from the first part of the protocol. Combined annealed RNA-RT reaction mix was then incubated at 23° C. for 10 minutes, 53° C. for 10 minutes, 80° C. for 10 min. The resulting 20 μl cDNA was then purified using Ethanol precipitation.

Ethanol Precipitation

Ethanol precipitation was used to concentrate the cDNA from Reverse Transcription and remove potential downstream inhibitors. The choice of appropriate salt was made based on which preparation would give maximum yield without inhibition of polymerase activity and removal of dNTPs. Consequently, 3 M, pH 5.2 sodium acetate (Invitrogen) was used as the salt. Reverse transcription product cDNA (20 μl) was combined with 0.1 volume of 3 M sodium acetate (i.e., 2.0 μl) and 1.0 μl of 20 mg/ml glycogen (ThermoFisher). Further, 3 volumes (i.e., 60 μl) of ice-cold, 100% ethanol was added and mixed well. The ethanolic solution was stored at −20° C. for 1 h, as determined by optimization experiments. Following this, cDNA was recovered by centrifugation at 16,000 g for 30 min at 4° C. The supernatant was carefully aspirated without disturbing the pellet. Subsequently, the pellet was washed with 0.5 ml of ice-cold, freshly prepared 70% ethanol. This was followed by centrifugation at maximum speed for 10 min at 4° C. The supernatant was removed, and the tube was left open at room temperature to ensure that last traces of fluid have evaporated. The pellet was then dissolved and resuspended in 6 μl of nuclease free water (Invitrogen).

Primers and Probes

Through the course of optimization and preliminary testing, at least ten different primer pairs specific to EGFRvIII deletion detection were designed and tested (Table A). The primer sets generated amplicons of varying lengths, ranging from 34 bp to 428 bp. The key criteria in selecting the optimal primer set included target amplicon size <100 bp, minimum formation of primer dimers, and absence of non-specific products. Tools used to design and assess different parameters include Primer-BLAST, Serial Cloner and Primer3Plus. Different primer sets were also compared based on GC content, Tₘ(° C.) mismatch, self-dimerization and hairpin formation. The final primer set selected for further optimization produced a target amplicon of 96 bp. Sequence for final choice of exon-exon spanning primers and probe are as follows: Forward mRNA primer (5′-GGCTCTGGAGGAAAAGAAAGGTAATT-3′; SEQ ID NO:8), Reverse mRNA primer (5′-CCGTCTTCCTCCATCTCATAGC-3′; SEQ ID NO:9), EGFRvIII mutant probe (5′-FAM-TGACAGATCACGGCTC-MGBNFQ-3′; SEQ ID NO:10). All oligonucleotide preparations were synthesized by ThermoFisher Scientific. Primers were purified by desalting and resuspended in DEPC-treated water, at a final concentration of 100 μM. Probe was HPLC purified and resuspended in 1×TE 100 μmol/μl. GAPDH Assay ID: Hs99999905_ml.

TABLE A
Primers for amplification and detection of EGFRvIII

| | | | | | Hetero | |
|---|---|---|---|---|---|---|
| | | | | GC | dimer/ | Amplicon |
| Forward | Reverse | Tm | | content | kcal/ | size |
| Primer | Primer | (° C.) | Length | (%) | mole | (bp) |
| GGCTCT | CCGTCTTCCT | Fwd: | Fwd: 26 | Fwd: | −5.49/ | 96 |
| GGAGG | CCATCTCATA | 57.1 | Rev: 22 | 42% | −49.05 | |
| AAAAG | GC (SEQ ID | Rev: | | Rev: | | |
| AAAGG | NO: 9) | 57.1 | | 52% | | |
| TAATT | | | | | | |
| (SEQ ID | | | | | | |
| NO: 8 | | | | | | |
| GGACGA | TTCTTTTCCTC | Fwd: | Fwd: 17 | Fwd: | −6.21/ | 219 |
| CAGGCC | CAGAGCCCG | 58.4 | Rev: 20 | 70.6 | −41.45 | |
| ACCTC | (SEQ ID | Rev: | | Rev: | | |
| (SEQ ID | NO: 12) | 57.1 | | 55 | | |
| NO: 11) | | | | | | |
| GGACGA | CCGCAAGTGT | Fwd: | Fwd: 17 | Fwd: | −3.61/ | 320 |
| CAGGCC | AAGAAGTGCG | 58.4 | Rev: 20 | 70.6 | −39.62 | |
| ACCTC | (SEQ ID | Rev: | | Rev: | | |
| (SEQ ID | NO: 14) | 56.6 | | 55 | | |
| NO: 13) | | | | | | |
| GGACGA | CCATCAGTGG | Fwd: | Fwd: 17 | Fwd: | −9.5/ | 441 |
| CAGGCC | CGATCTCCA | 58.4 | Rev: 19 | 70.6 | −37.5 | |
| ACCTC | (SEQ ID | Rev: | | Rev: | | |
| (SEQ ID | NO: 16) | 57 | | 57.9 | | |
| NO: 15) | | | | | | |
| CTCCTGG | GAGCCGTGAT | Fwd: | Fwd: 65.9 | Fwd: | −6.21/ | 86 |
| CGCTGCT | CTGTCACCAC | 16 | Rev: 65.4 | 75 | −37.04 | |
| GG (SEQ | (SEQ ID | Rev: | | Rev: | | |
| ID NO: 17) | NO: 18) | | | 60 | | |
| CGGCGAG | CATCTCATAGC | Fwd: | Fwd: 67.0 | Fwd: | −9.28/ | 95 |
| TCGGGCT | TGTCGGCCC | 16 | Rev: 65.1 | 75 | −39.73 | |
| CT (SEQ | (SEQ ID | Rev: | | Rev: | | |
| ID NO: 19) | NO: 20) | 20 | | 60 | | |
| CTGGCTG | GGACGCACGA | Fwd: | Fwd: 66.5 | Fwd: | −8.7/ | 82 |
| CGCTCTG | GCCGTGATC | 16 | Rev: 68.0 | 75 | −40.56 | |
| CC (SEQ | (SEQ ID | Rev: | | Rev: | | |
| ID NO: 21) | NO: 22) | 19 | | 68.4 | | |
| GAGTCGG | CCACAGGCTC | Fwd: | Fwd: 66.9 | Fwd: | −5/ | 72 |
| GCTCTGG | GGACGCAC | 22 | Rev: 68.8 | 59.1 | −44.02 | |
| AGGAAA | (SEQ ID | Rev: | | Rev: | | |
| AG (SEQ | NO: 24) | 18 | | 72.2 | | |
| ID NO: 23) | | | | | | |
| TCGGGCT | CTTCCTCCATC | Fwd: | Fwd: 63.5 | Fwd: | −14.79/ | 95 |
| CTGGAGG | TCATAGCTGTC | 17 | Rev: 62.6 | 58.8 | −38.2 | |
| AAA | (SEQ ID | Rev: | | Rev: | | |
| (SEQ ID | NO: 26) | 22 | | 50% | | |
| NO: 25) | | | | | | |
| CTTCGGG | ACCAATACCT | Fwd: | Fwd: 22 | Fwd: | −6.68/ | 243 |
| GAGCAGC | ATTCCGTTACA | 71.5 | Rev: 22 | 68.2 | −49.22 | |
| GATGCGA | C (SEQ ID | Rev: | | Rev: | | |
| C (SEQ | NO: 28) | 61 | | 40.9 | | |
| ID NO: 27) | | | | | | |
| ATGCGAC | ACCAATACCT | Fwd: | Fwd: 20 | Fwd: | −8.02/ | 228 |
| CCTCCGG | ATTCCGTTACA | 67.3 | Rev: 22 | 75 | −47.96 | |
| GACGGC | C (SEQ ID | Rev: | | Rev: | | |
| (SEQ ID | NO: 30) | 52.1 | | 40.9 | | |
| NO: 29) | | | | | | |
| ATGCGAC | GAGTATGTGT | Fwd: | Fwd: 18 | Fwd: | −6.24/ | 352 |
| CCTCCGG | GAAGGAGT | 62.8 | Rev: 18 | 72.2 | −421.76 | |
| GACG | (SEQ ID | Rev: | | Rev: | | |
| (SEQ ID | NO: 32) | 48.1 | | 44.4 | | |
| NO: 31) | | | | | | |
| CCAGTAT | TCAGAATATC | Fwd: | Fwd: 20 | Fwd: | −5.02/ | 431 |
| TGATCGG | CAGTTCCTGTG | 62.7 | Rev: 22 | 55 | −38.6 | |
| GAGAGC | G (SEQ ID | Rev: | | Rev: | | |
| (SEQ ID | NO: 34) | 62.6 | | 45.5 | | |
| NO: 33) | | | | | | |

Optimized Droplet Digital PCR (ddPCR) Assay in Tumor Tissue and Cell Lines

Droplet digital PCR (ddPCR) was performed using purified cDNA obtained from reverse transcription of RNA isolated from cell lines and tumor tissue. The ddPCR reaction was performed in the Applied Biosystems™ 96-well Thermal Cycler (ThermoFisher Scientific) in a final volume of 20 μl. The optimized reaction mixture was prepared using 10 μl of ddPCR Supermix for Probes (no dUTP) (Bio-Rad), 1500 nM forward and reverse primers as shown above and 350 nM probe, and 2 μl of 5 M Betaine solution (Sigma-Aldrich). The prepared reaction mixture was transferred to the wells of DG8 cartridge. To generate the droplets, 70 μl of droplet generation oil for probes (Bio-Rad) was added and the plate was loaded into the QX200 Droplet generator (BioRad). The droplet emulsions were then carefully transferred to a semi-skirted, PCR-clean 96 well plate (Eppendorf) using a multichannel pipette. Lastly, the plate was sealed using PX1 PCR plate sealer (Bio-Rad). Based on the annealing temperature gradient optimization experiment, the final thermal cycling conditions were as follows: initial enzyme activation at 95° C. (51% ramp) for 5 minutes, then 40 cycles of denaturation at 94° C. (51% ramp) for 30 seconds, and annealing/extension at 55.5° C. for 1 minutes, followed by enzyme inactivation at 98° C. for 10 min and final hold at 4° C. until analysis. Droplets were analyzed using QX 200 droplet reader (Bio-Rad) and data were acquired and analyzed with QuantaSoft analysis software (Bio-Rad).

Optimized Quantitative (qPCR) Assay in tumor tissue and cell lines Quantitative PCR (qPCR) was performed using purified cDNA obtained from reverse transcription of RNA isolated from cell lines and tumor tissue. The qPCR reaction was performed in the Applied Biosystems' StepOnePlus™ Real-Time PCR (ThermoFisher Scientific) in a final volume of 20 μl. The optimized reaction mixture was prepared using 10 μl of TaqMan™ Universal Master Mix II, no UNG (ThermoFisher Scientific), 1500 nM forward and reverse primers as shown above and 350 nM probe, and 2 μl of 5 M Betaine solution (Sigma-Aldrich). The reaction mixture was prepared in the wells of 96 well, non-skirted PCR plate (ThermoFisher Scientific). Lastly, the plate was sealed using the Adhesive PCR plate sealer (ThermoFisher Scientific). Based on the annealing temperature gradient optimization experiment, the final thermal cycling conditions were as follows: initial enzyme activation at 95° C. (100% ramp) for 5 minutes, then 40 cycles of denaturation at 94° C. (100% ramp) for 30 seconds, annealing at 55.5° C. for 30 sseconds, and extension at 60.0° C. for 1 min. Quantitative analysis was performed using Comparative Ct (ΔΔCt) method with StepOne™ Software (Applied Biosystems, ThermoFisher Scientific). Data were acquired and interpreted using Amplification plots. To calculate amplification efficiency, Ct values were plotted on a logarithmic scale along with corresponding concentrations of RNA input. Next, a linear regression curve through the data points was generated and slope of the trend line calculated. Finally, efficiency was calculated using the equation: E=−1+10(−1/slope).

Optimized Droplet Digital PCR (ddPCR) Assay in Plasma and CSF Derived Extracellular Vesicles (EVs).

Droplet digital PCR (ddPCR) was performed using purified cDNA obtained from reverse transcription of EV RNA isolated from plasma and CSF samples. For plasma samples, detection of low levels of mutant EV RNA was enhanced via PCR pre-amplification. The pre-amplification ddPCR was performed in the Applied Biosystemsm 96-well Thermal Cycler (ThermoFisher Scientific) in a final volume of 20 μl. The optimized reaction mixture was prepared using 6 μl of purified cDNA, 10 μl of ddPCR Supermix for Probes (no dUTP) (Bio-Rad), 1500 nM forward and reverse primers, and 1.0 μl of 10 mM dNTP (ThermoFisher). The thermocycling conditions were as follows: initial enzyme activation at 95° C. (51% ramp) for 5 minutes, then 20 cycles of denaturation at 94° C. (51% ramp) for 30 seconds, and annealing/extension at 55.5° C. for 1 minutes, followed by enzyme inactivation at 98° C. for 10 min. The final ddPCR was performed using the pre-amplified product in a final reaction volume of 20 μl. The reaction mixture was prepared using 2 μl of pre-amplified product, 10 μl of ddPCR Supermix for Probes (no dUTP) (Bio-Rad), 1500 nM forward and reverse primers and 350 nM probe, and 2 μl of 5 M Betaine solution (Sigma-Aldrich). The final thermal cycling conditions were as follows: initial enzyme activation at 95° C. (51% ramp) for 5 minutes, then 40 cycles of denaturation at 94° C. (51% ramp) for 30 seconds, and annealing/extension at 55.5° C. for 1 minutes, followed by enzyme inactivation at 98° C. for 10 min and final hold at 4° C. until analysis. Droplets were analyzed using QX 200 droplet reader (Bio-Rad) and data were acquired and analyzed with QuantaSoft analysis software (Bio-Rad).

Droplet Digital PCR (ddPCR) dMIQE 2020 Guideline Compliance

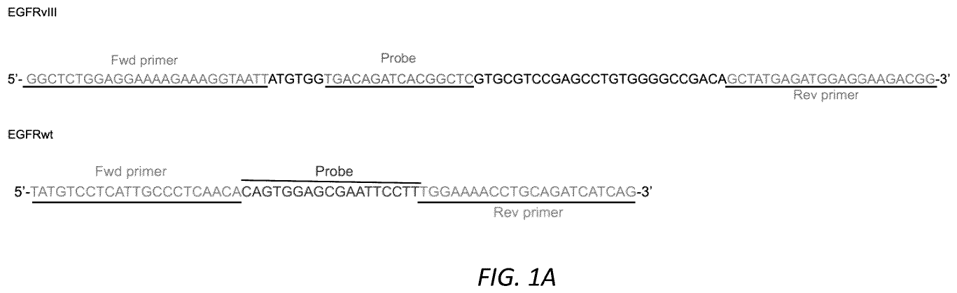

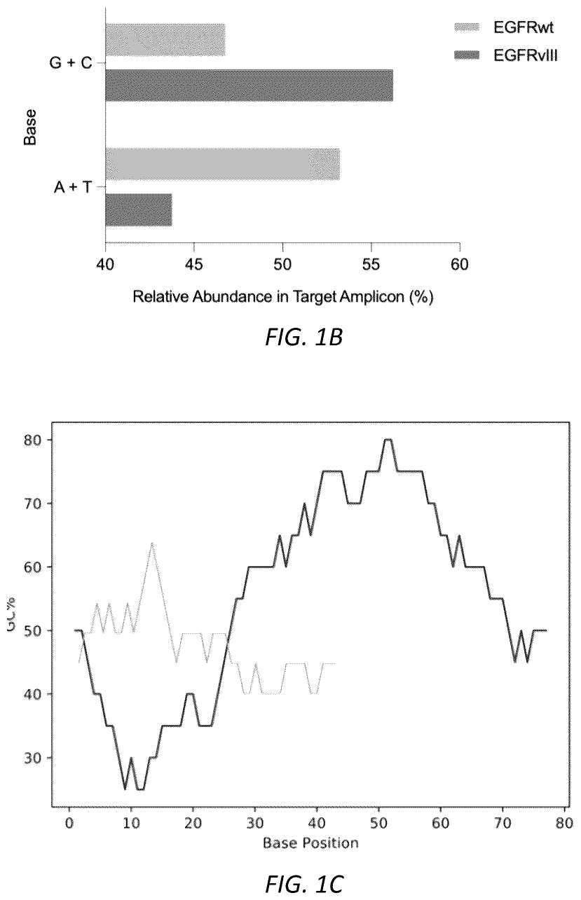

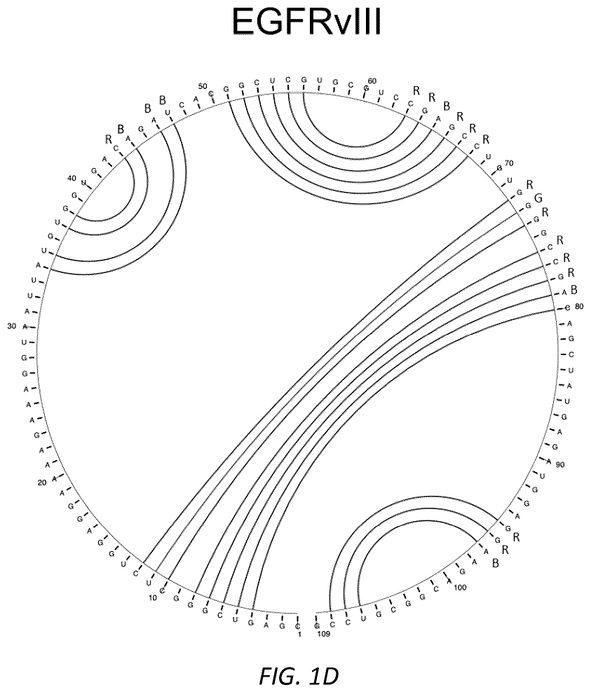

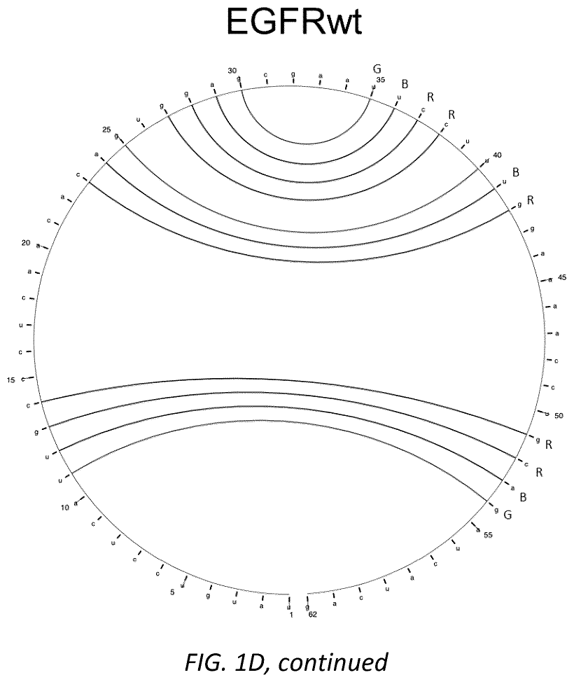

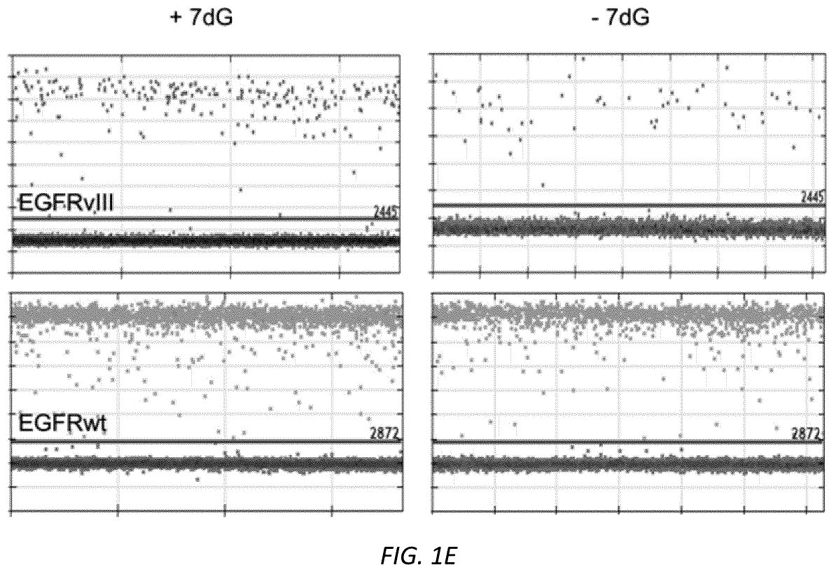

This section of the methods describes our compliance with the updated 2020 dMIQE Guidelines for the technical development of a plasma-based ddPCR assay for the EGFRvIII mutation detection. Specimen type numbers, sampling procedure, aliquotation, conditions and duration are provided in the following sections of the Methods: Study Population, Tumor Tissue Processing, Cell Lines, and Plasma Processing. Details about the specific Extraction techniques, Nucleic Acid Assessment/Storage, and Reverse Transcription are provided in sections: RNA Isolation from cell lines and tumor tissue, and Exosomal RNA Isolation from plasma samples. Information about the ddPCR Oligonucleotides and its target sequences are provided along with the protocol both in FIG. 1(b) and in the section entitled Primers and Probe.

Quantification of EGFRvIII Mutation in Plasma

The number of EGFRvIII copies per mL of plasma was calculated from QuantaSoft data as follows: Copies/mL plasma=C EV/TV/P where C=copies per 20 μL, EV=exNA elution volume (μL), TV=exNA input into ddPCR reaction (μL) and p=plasma volume (mL). Only samples with >10,000 droplets/well were included in the analysis. A positive sample is considered one with at least 1 event on at least two wells. Four wells are analyzed in parallel for each sample.

Statistical Analysis

Statistical analysis was performed using the unpaired two-tailed Student's t-test in GraphPad Prism 8 software and p<0.05 was statistically significant. The results are presented as mean±SD.

Institutional Review Board Statement

Our studies were conducted in accordance with principles for human experimentation as defined in the U.S. Common Rule and were approved by the Human Investigational Review Board of each study center under a Partners institutional review board (IRB)-approved protocol. All healthy control subjects were screened for pertinent oncologic and neurologic medical histories. Individuals with a history of cancer, neurological disorders, and infectious diseases were excluded from the study.

Informed Consent Statement

All samples were collected with written informed consent after the patient was advised of the potential risks and benefits, as well as the investigational nature of the study.

### Example 1. EGFRvIII mRNA is Rich in GC Regions and G Repeats Leading to Secondary Structures

Epidermal growth factor receptor variant III (EGFRvIII) is a result of an 801-bp deletion in the extracellular domain of the EGFR gene (exons 2-7). Custom primers and TaqMan probes were designed for targeted detection of a 96-bp amplicon in EGFRvIII and a 62-bp amplicon in EGFRwt mRNA sequence and used in all experiments (FIG. 1a). The composition of nitrogenous bases in the EGFRvIII amplicon was compared to the EGFRwt amplicon. To account for the difference in amplicon length, relative abundance of different bases was calculated as a percentage. Interestingly, the GC content of EGFRvIII amplicon was 1.2-fold higher compared to the counterpart EGFRwt (56% and 46% respectively, (FIG. 1b). Further comparison of the GC content (%) distribution across the length of two target amplicons was performed by calculating average GC content of smaller segments (20-bp) in each RNA sequence. This revealed segments within the EGFRvIII amplicon with GC content as high as 80%. Furthermore, the majority sequence of EGFRvIII amplicon had a GC content in the range of 60-80%. Conversely, the RNA sequence of EGFRwt amplicon using the same analytical parameters had a lower GC content (40-50%) with only one peak seen at 60% (FIG. 1c).

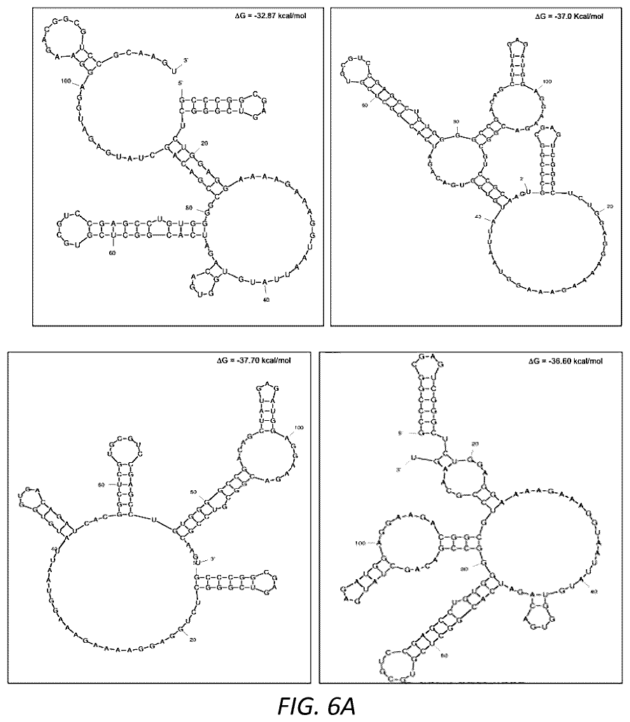

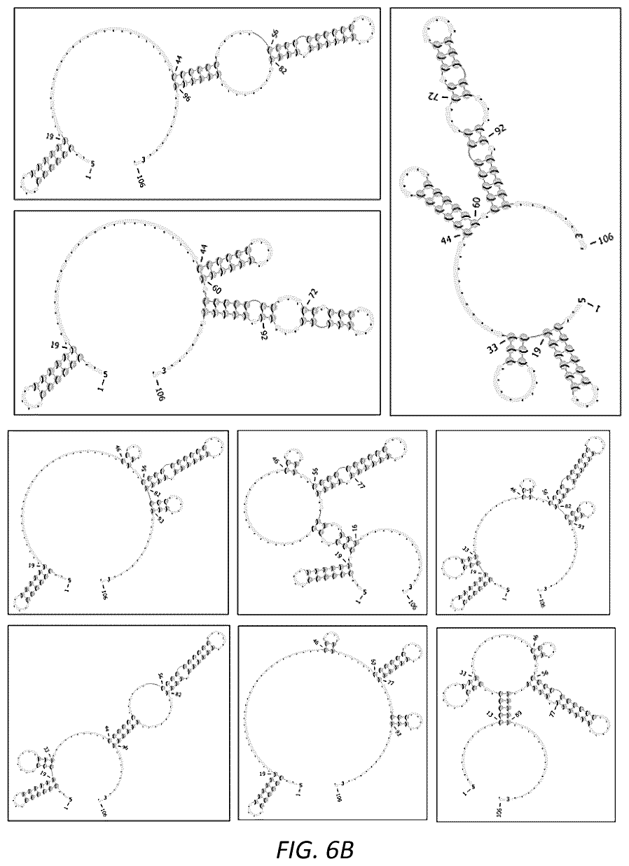

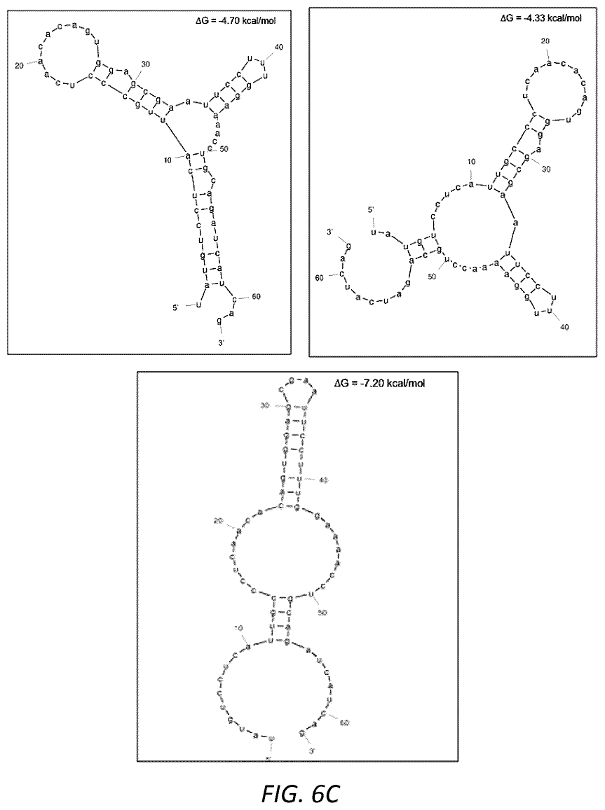

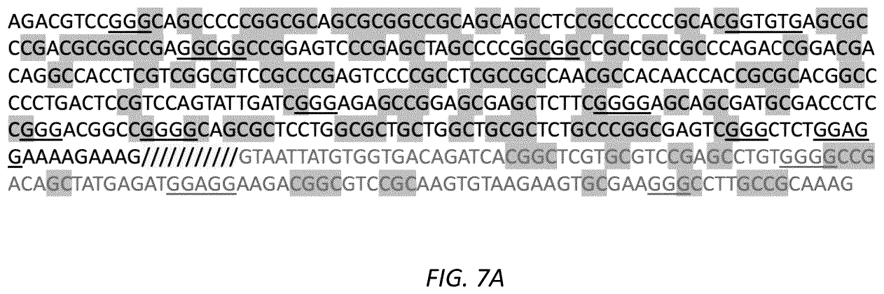

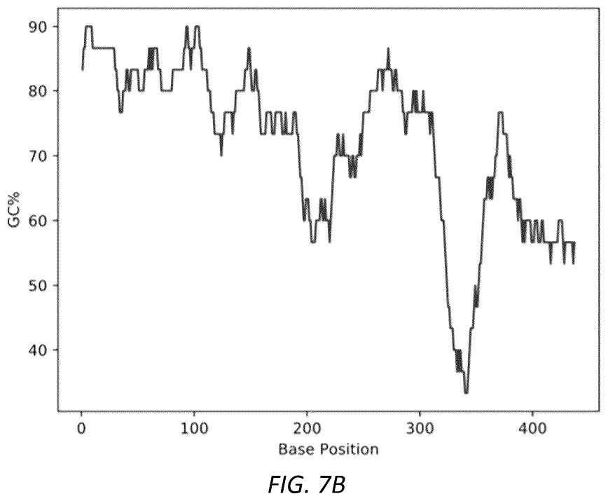

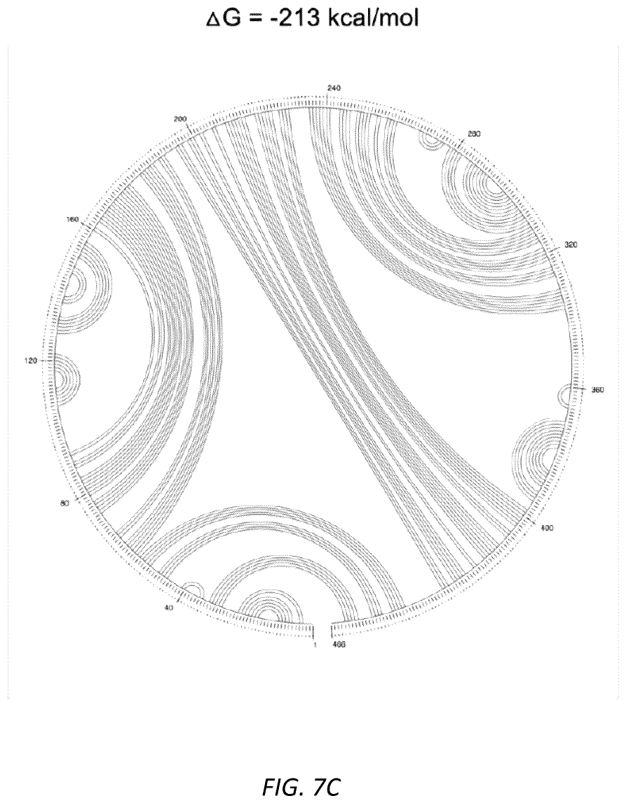

High GC content is one of the factors contributing to secondary structure formation. To further understand the structure we used Mfold^{21,22}, an RNA folding prediction tool to visualize potential secondary structures in the target amplicons. The software is based on minimum free energy (MFE) algorithms that consider the complex interactions between RNA secondary structures and free energy. We determined four distinct Minimum Free Energy (MFE) secondary structures in EGFRvIII amplicon with ΔG (Gibbs free energy) ranging from −32.9 kcal/mol to −37.7 kcal/mol, while the EGFRwt amplicon had only three foldings with a significantly lower ΔG between −4.3 kcal/mol and −7.2 kcal/mol, suggesting a higher thermostability of 3D secondary structures for EGFRvIII RNA (FIGS. 6a-c). Furthermore, the circle graphs for both EGFRvIII (FIG. 1d, left panel) and EGFRwt (FIG. 1d, right panel) indicate individual base pair interactions instrumental in stabilizing the predicted secondary structures. This is represented by an arc each indicating a distinct base pair (Watson Crick and non-Watson Crick) interaction. This phenomenon also contributes to the formation of predicted pseudoknots in EGFRvIII amplicon (FIG. 6b). Overall, the predicted three-dimensional secondary structures are thermodynamically more stable in EGFRvIII (ΔG: EGFRvIII−30.7 kcal/mol, EGFRwt −7.2 kcal/mol) compared to EGFRwt. To investigate this further, the RNA sequence unique to EGFRvIII (exon 1:exon 8, 467-bp) was also analyzed via Mfold using the same metrics. This analysis further revealed the presence of close to 90% of GC or G repeat region (FIGS. 7a,b) and at least twenty distinct secondary structures with a high ΔG (approximately, −200 kcal/mol) indicating high thermal stability of the predicted foldings (FIG. 7b-d).

Based on these results, we examined the potential of using 7-deaza-2′-deoxyguanosine 5′-triphosphate (7 dG) to overcome the negative impact on downstream analysis of this higher-level organization. Reverse transcription and droplet digital PCR (ddPCR) amplification of EGFRvIII and EGFRwt target amplicons was performed with and without the addition of 7 dG. We observed that addition of the additive resulted in a notable improvement in the amplification of EGFRvIII cDNA with higher number of EGFRvIII copies and no significant effect on the amplification of EGFRwt (FIG. 1e).

### Example 2. Droplet Digital PCR Assay Design and Optimization

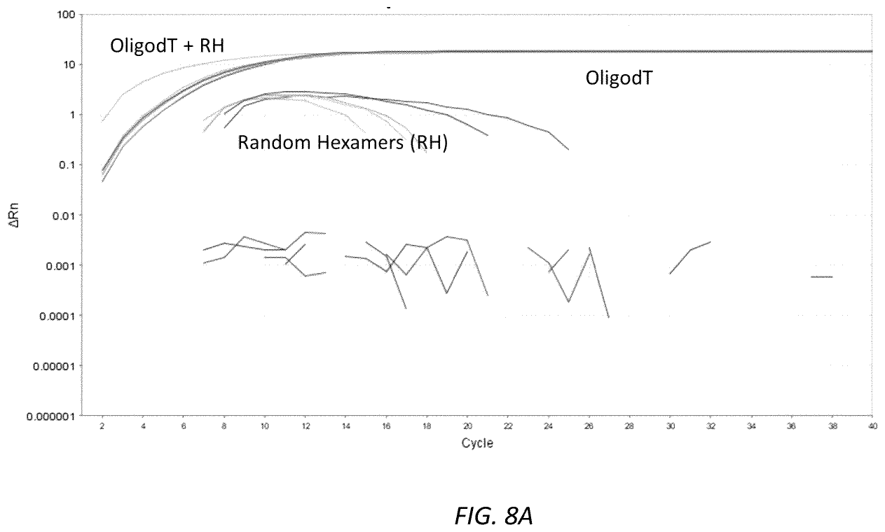

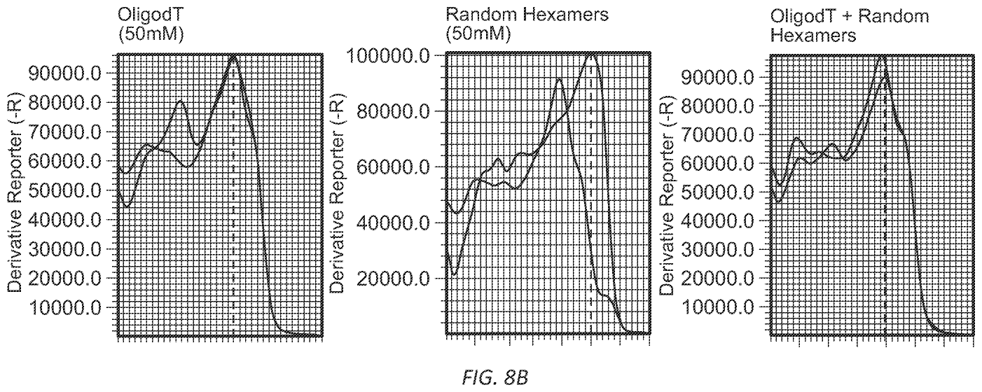

One of the main barriers to successful clinical implementation of EV-based liquid biopsy for brain tumors is the significantly low concentration of mutant EV RNA isolated from circulating EVs in patient plasma combined with the high signal-noise ratio typical of a complex biofluid such as plasma. The goal was to maximize the sensitivity of the assay in patient plasma derived EV RNA and minimize non-specific signal using an optimal combination of different measures. We first optimized the Reverse Transcription reaction by testing a series of concentrations of Oligo d(T)₂₀ and Random hexamers (FIGS. 8A-B) with the final concentration set at 50 μM each of both Oligo d(T)₂₀ and Random hexamers.

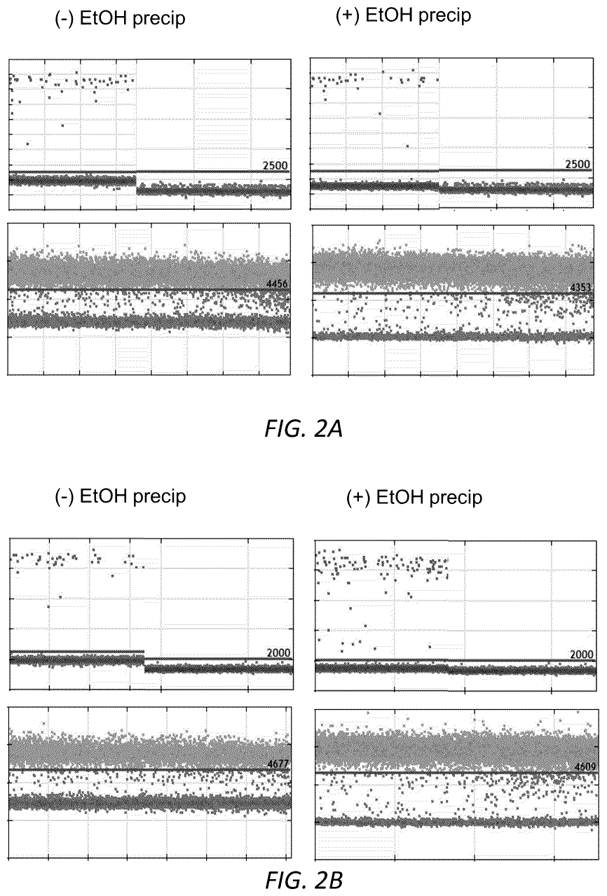

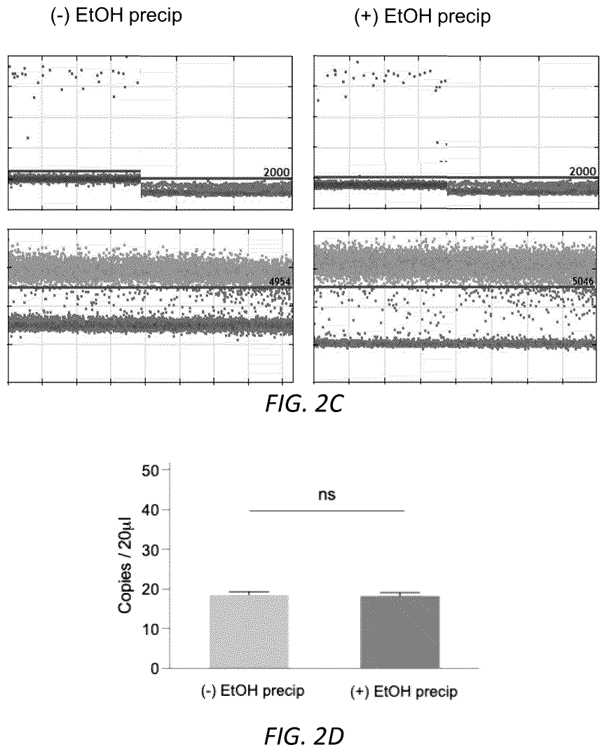

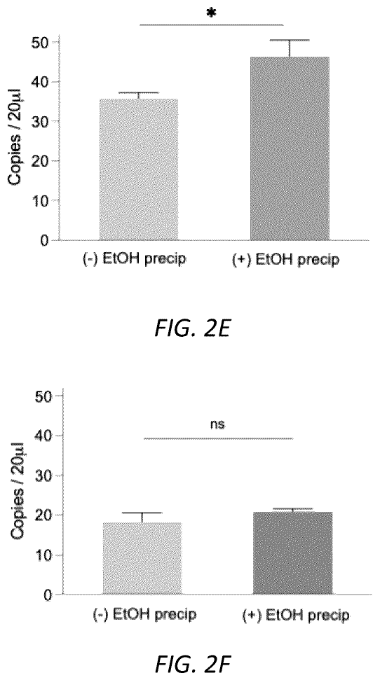

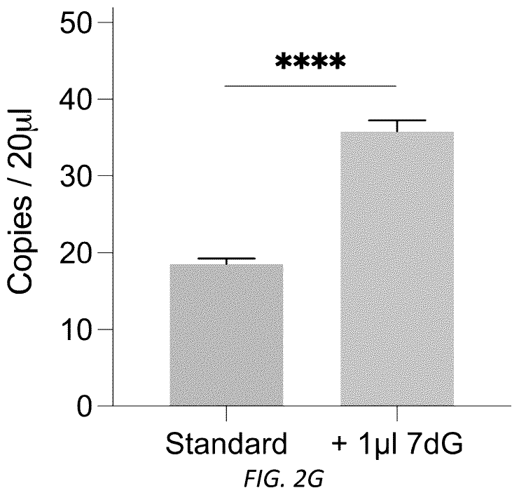

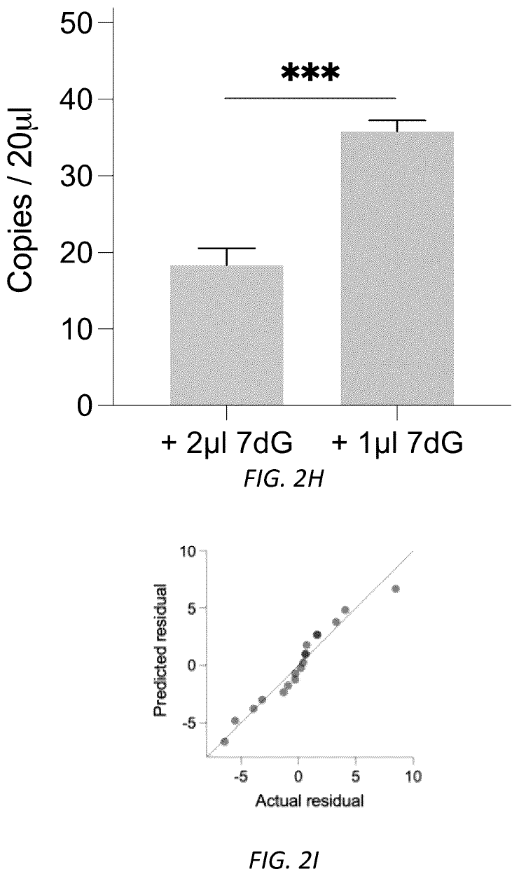

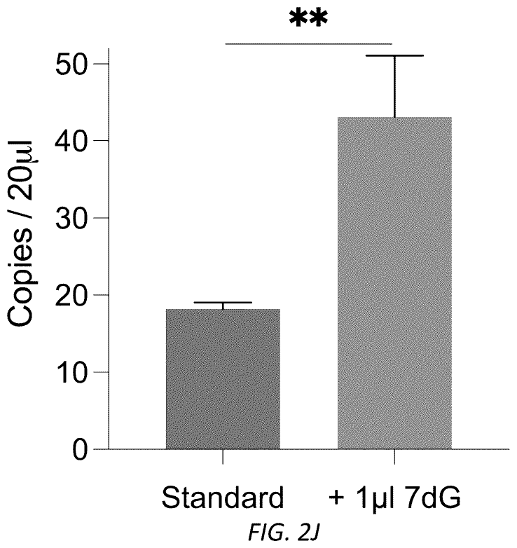

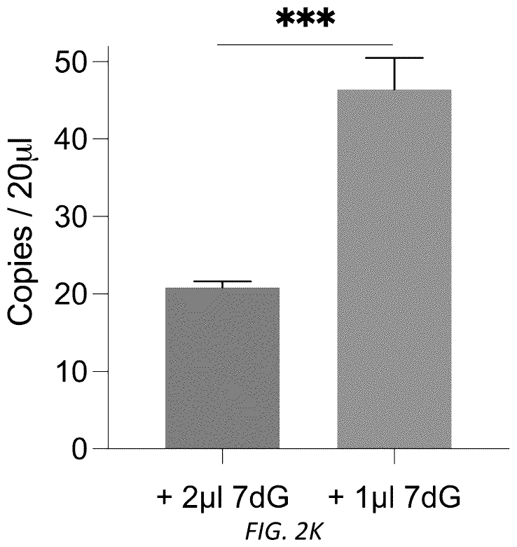

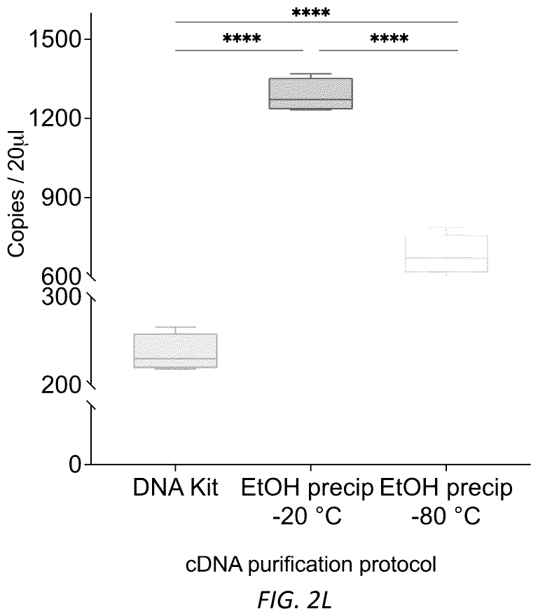

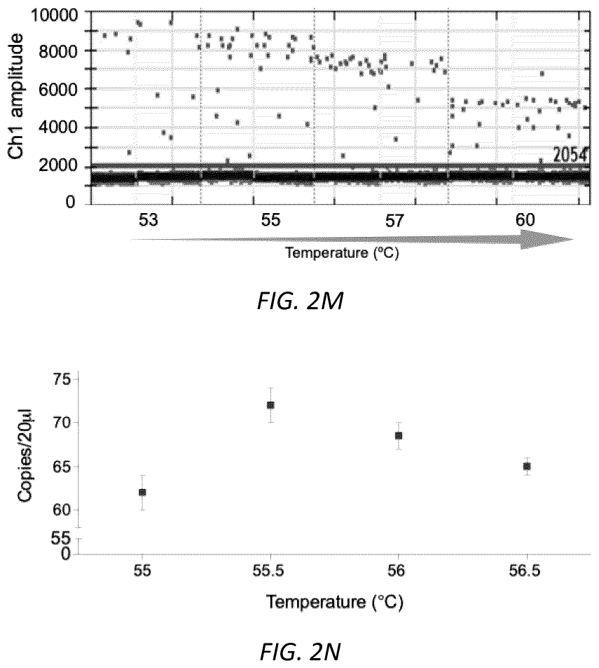

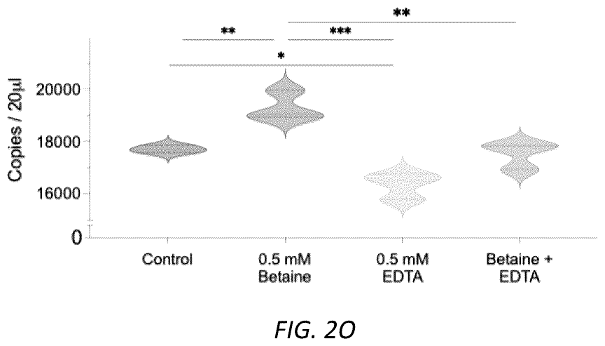

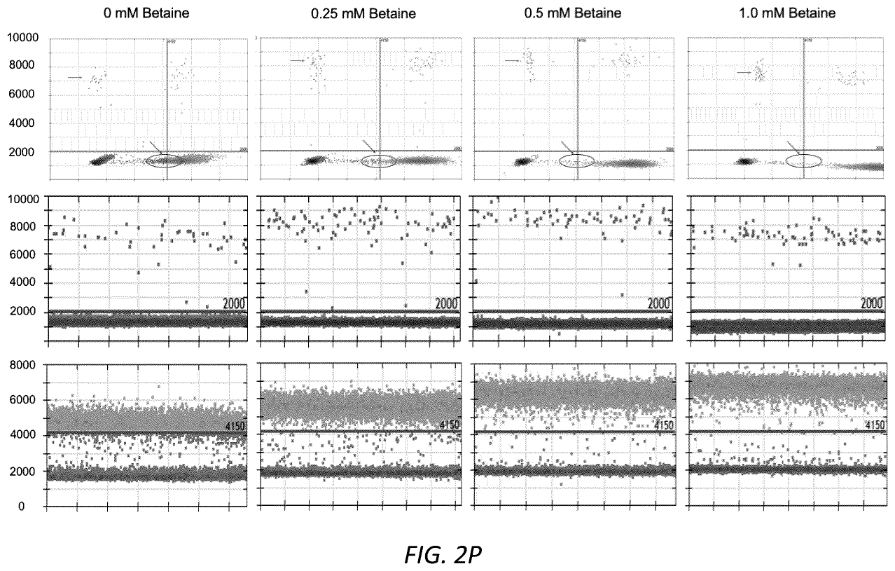

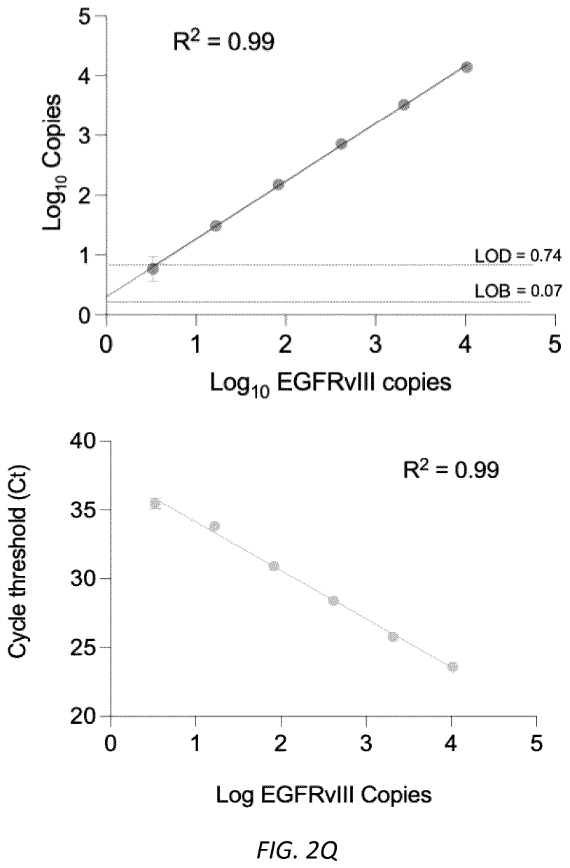

This was followed by testing the effect of addition of increasing amounts (1 μl or 2 μl) of 7 dG at the Reverse transcription (RT) step. Total RNA extracted from EGFRvIII tumor tissue, was diluted to 1 ng and used as a starting input for three different reverse transcription conditions (two replicates per condition) in a reaction volume of 20 μl; Standard (mock 7 dG), Standard+1 μl 7 dG, Standard+2 μl 7 dG. The resulting cDNA from one replicate of each condition was subject to ethanol precipitation and resuspended in 20 μl of nuclease free water prior to ddPCR amplification. The second replicate was run in parallel without ethanol precipitation. Across all RT conditions when cDNA was not further purified by ethanol precipitation, we observed an elevation of baseline in the positive well, located above the baseline of no template control (NTC) (FIG. 2 a,b,c, left panel). On the other hand, post ethanol precipitation, this inhibition was overcome leading to a significant improvement in the baseline correction and alignment to the reference NTC well (FIG. 2 a,b,c, right panel). Again this effect was observed across all three RT conditions. A similar trend was also seen in GAPDH which was used as a reference gene in channel 2 (FIG. 2 a,b,c,). This therefore, highlighted the role of potential ddPCR inhibitors which can be removed using ethanol precipitation.

In addition, comparison of the ddPCR results across the three RT conditions continued to demonstrate the favorable effect of adding 7 dG. RT preparation with 1 μl 7 dG resulted in the highest number of EGFRvIII copies with a statistically significant higher detection post ethanol precipitation of 7 dG treated cDNA (FIG. 2 d,e,f). Furthermore, we report a statistically significant increase in the number of mutant copies with the addition of 1 μl, as opposed to 2 μl, of 7 dG at the RT step (FIG. 2 g, j, h, k). The significantly higher yield of EGFRvIII copies post ethanol precipitation (FIG. 2e) pointed to the potential carryover and inhibitory effect of excess 7 dG at the RT and PCR level. All ddPCR conditions were run in triplicates and the results of ANOVA across all conditions were statistically significant, at R² value 0.98 (FIG. 2i). We also compared several clean up methods including ethanol precipitation and commercial kits and determined the highest recovery of EGFRvIII mRNA using ethanol precipitation at −20° C. (FIG. 2l).

To achieve maximum cluster separation on ddPCR, a temperature gradient was conducted (53° C. to 60° C.) to determine the optimal annealing temperature (FIG. 2m). Better cluster separation and tightly formed droplets were seen at lower as compared to higher temperature. The fluorescence of the mutant signal was observed to decrease significantly in amplitude after 57° C. Furthermore, at temperature <55° C. the clusters were more diffuse. To investigate this further a second round of temperature gradient was conducted at 0.5° C. increment within the optimal range (55° C. to 56.5° C.) determined from the preliminary findings (FIG. 2n). Based on results of the second temperature gradient, 55.5° C. was selected as the optimal annealing temperature, since it resulted in best cluster separation and maximum number of tightly formed mutant droplets (FIG. 2n).

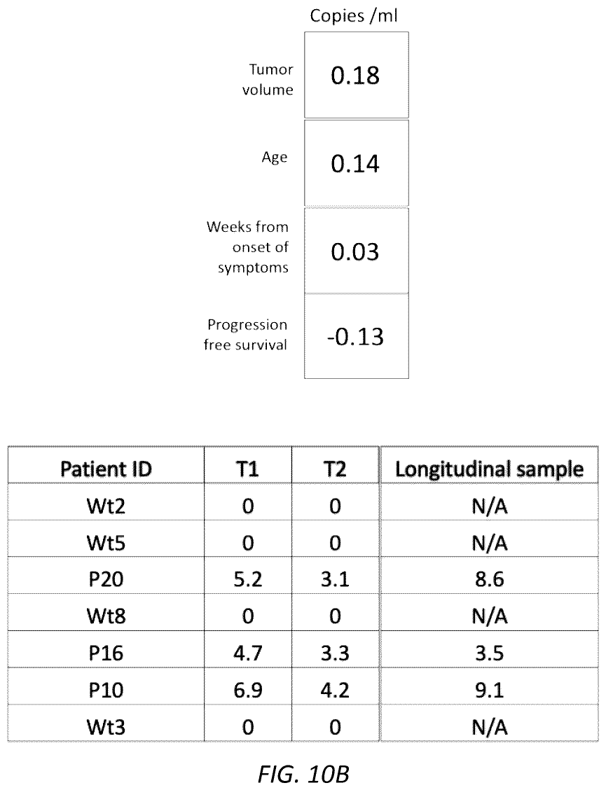

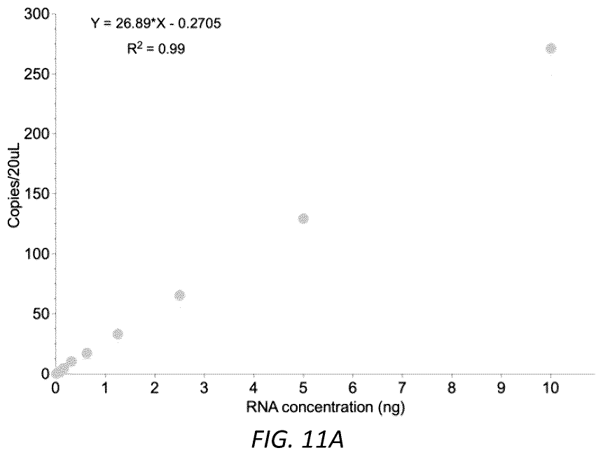

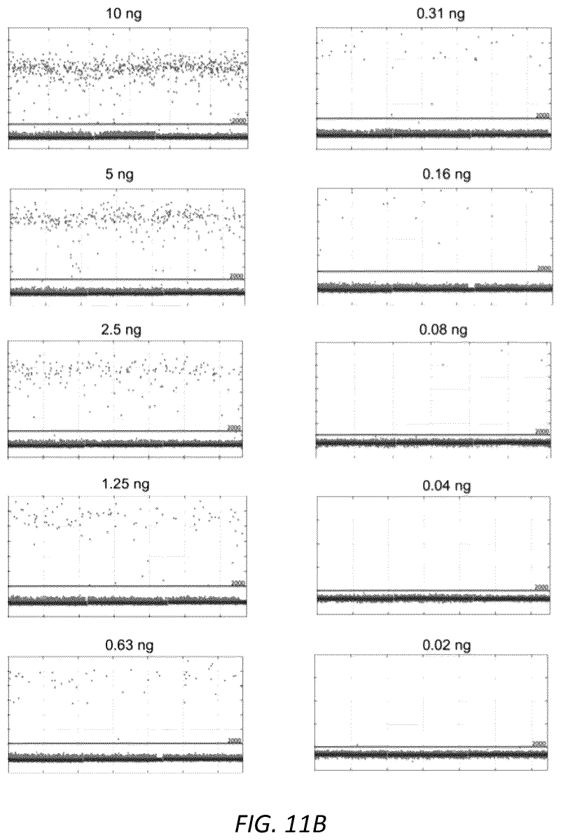

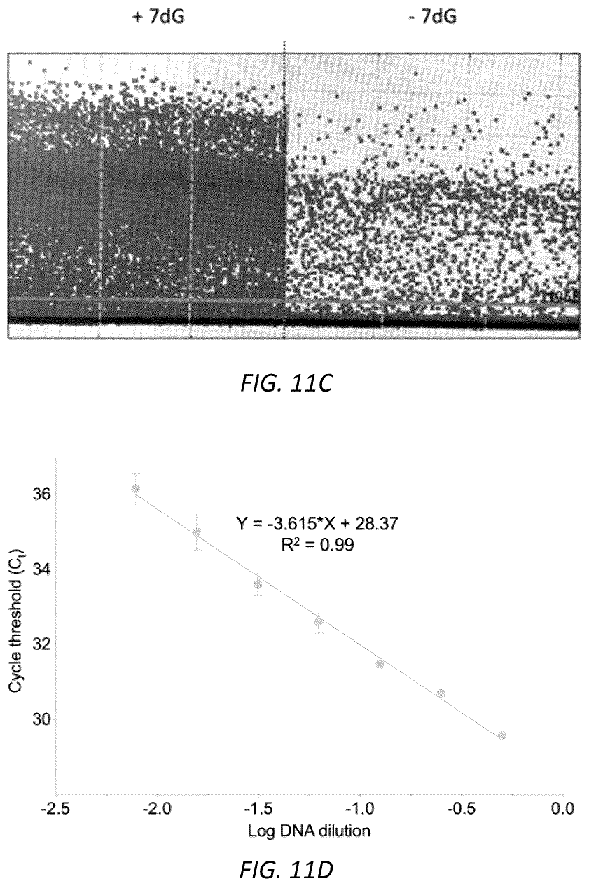

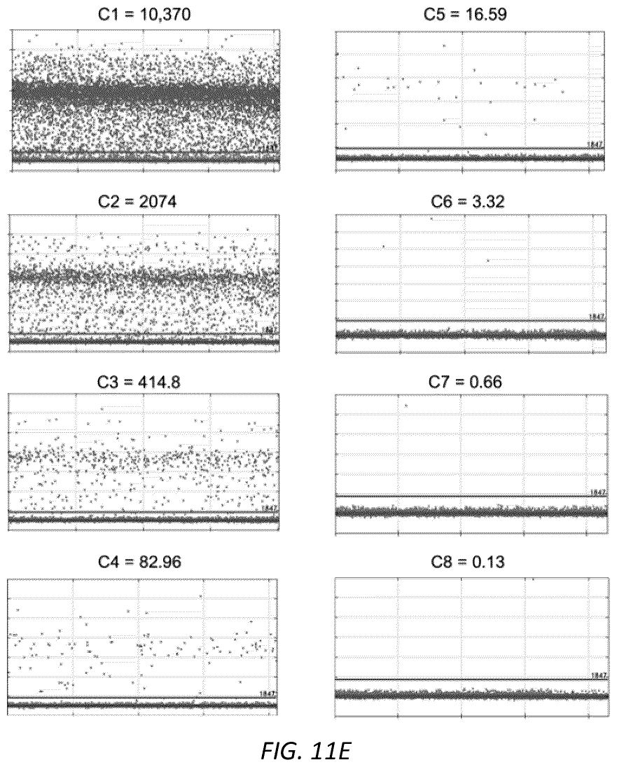

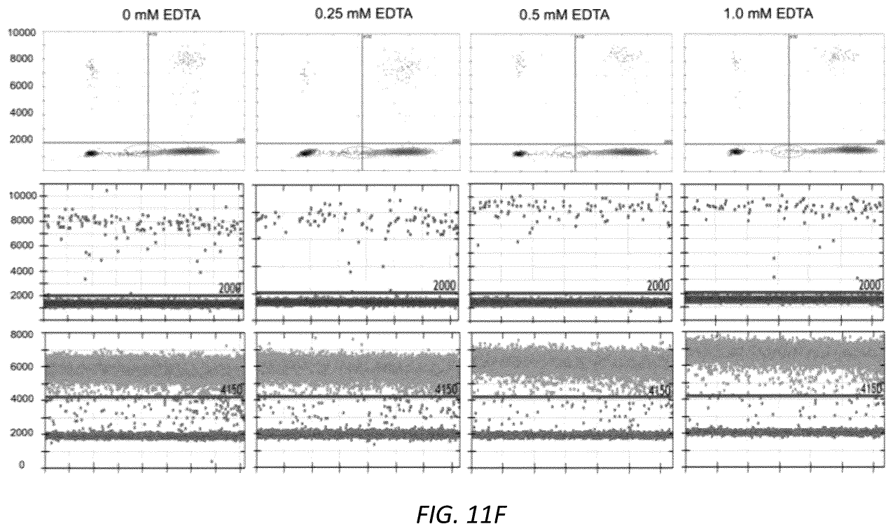

Lastly, we also explored the effect of employing different ddPCR additives: Betaine (FIG. 2p) and Ethylenediaminetetraacetic acid (EDTA; FIG. 11f) at serial dilutions (0.25 mM, 0.5 mM and 1.0 mM). The analysis demonstrated a significant improvement in mutant cluster separation at 0.5 mM concentration each of EDTA and Betaine when compared to control (FIG. 11f and FIG. 2p, respectively). To determine the quantitative effect of the additives on measured EGFRvIII copies, 0.5 mM each of Betaine and EDTA were run individually and in combination (FIG. 2o). At the chosen concentration of 0.5 mM Betaine alone, the measured EGFRvIII concentration was consistently higher than all other conditions (FIG. 2o). Moreover, we continued to see a statistically significant decrease in mutant signal with the addition of 0.5 mM EDTA (both, individually and when used in combination with Betaine). Furthermore, as previously observed use of 0.5 mM Betaine concentration also improved the mutant cluster separation from baseline and cluster tightness as evidenced by increase in fluorescence amplitude on 1D and 2D ddPCR plots (FIG. 2p).

Optimized assay conditions were then evaluated using EGFRvIII synthetic RNA copies spiked into the RT reaction (See Methods). The resulting cDNA from each RNA concentration was amplified using ddPCR (top, FIG. 2q) and quantitative PCR (qPCR) (bottom, FIG. 2q). RT conversion is highly efficient and linear. For ddPCR we report analytical parameters including Limit of Detection (LOD, 5.53 copies of EGFRvIII) and Limit of Blank (LOB, 1.18 copies of EGFRvIII) (top, FIG. 2q, ddPCR 1D plots in FIG. 11e). For qPCR we report the linearity of Cycle threshold (Ct) across a range of EGFRvIII copies spike-in. Overall efficiency of qPCR is 92.2% (calculation described in methods). EGFRvIII synthetic RNA was also reverse transcribed into cDNA with and without 7 dG to determine the difference in detection potential by downstream ddPCR amplification. We show a significant difference in amplification efficiency when comparing 7 dG to no 7 dG (FIG. 11c). The experiments with serial dilution of tumor tissue RNA reverse transcribed into cDNA followed by PCR amplification were also run in parallel (FIG. 11 a, b, d). The final optimized assay was then ready to be applied to plasma derived EV RNA from glioma patients.

### Examples 3. Increased Prevalence of EGFRvIII mRNA Detected in Glioma Tumor Tissue Upon Addition of 7 dG

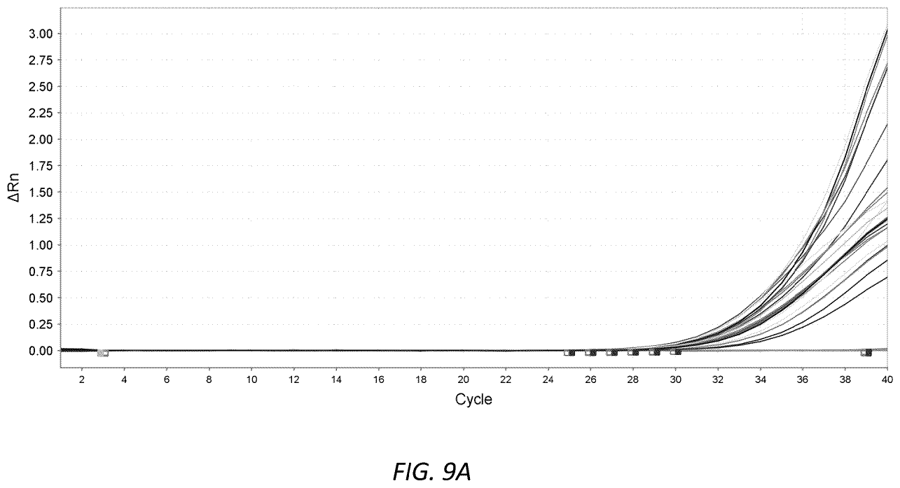

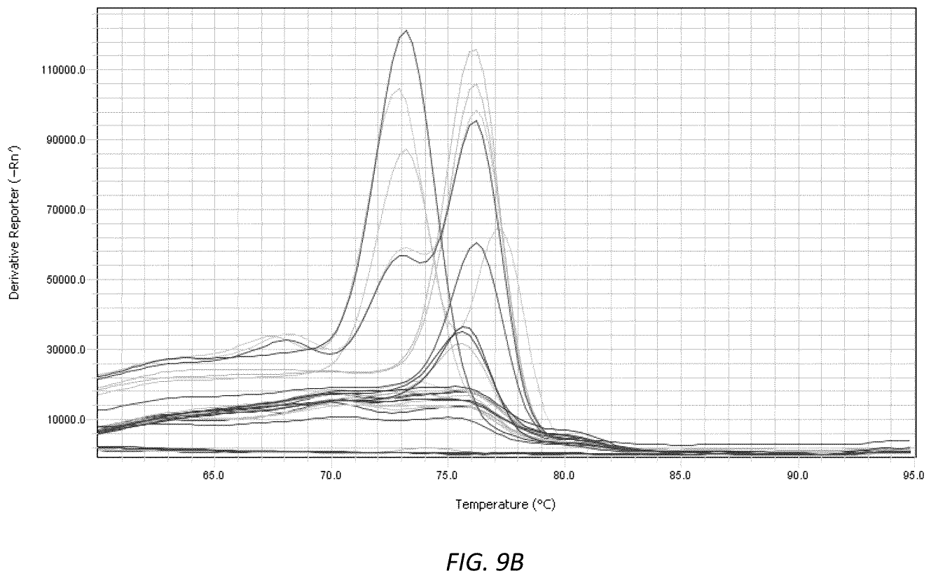

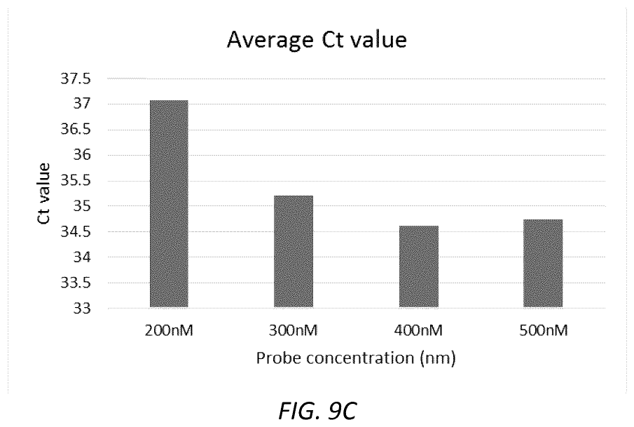

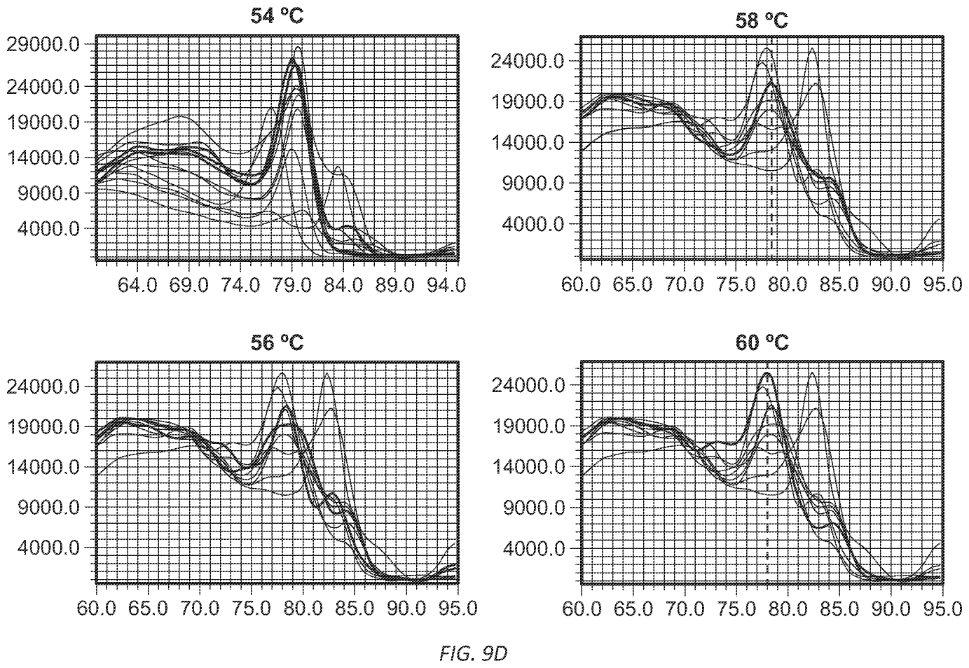

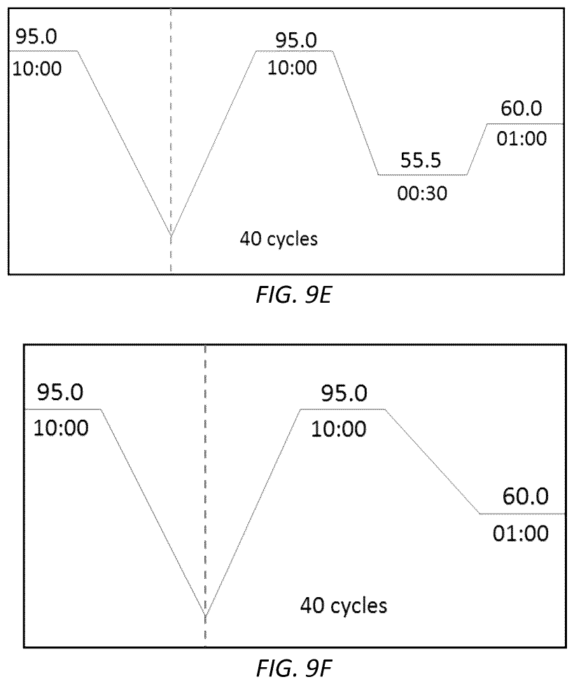

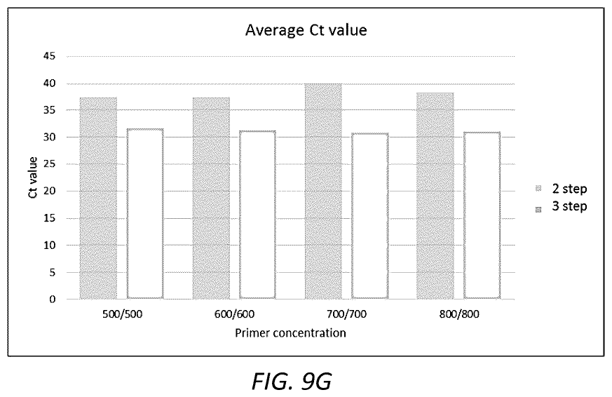

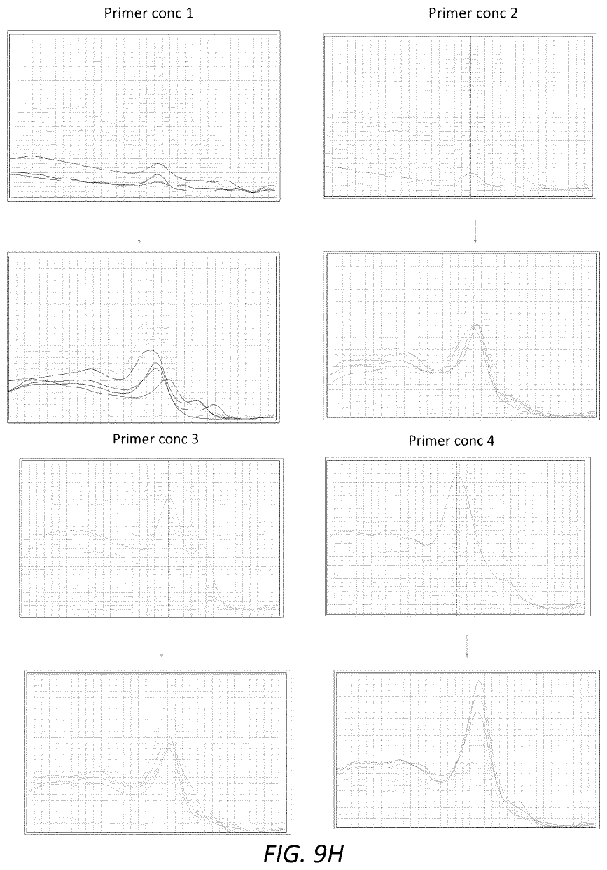

Having established the value of 7 dG addition, we sought to determine the assay performance in RNA extracted from tumor tissue using quantitative PCR (qPCR). Two separate cohorts with tissue availability were analyzed to test the rigor and reproducibility of the assay. We first determined optimal qPCR conditions for a range of variables, including, primers (FIG. 9a,b, Table A) and probe concentrations (optimal 300-400, e.g., 350) (FIG. 9c), annealing temperature gradient (optimal 53-60, e.g., 55.5) (FIG. 9d), and two step or three step cycling conditions (FIG. 9e,f,g,h). The final protocol with the lowest Ct value was used for tumor tissue testing.

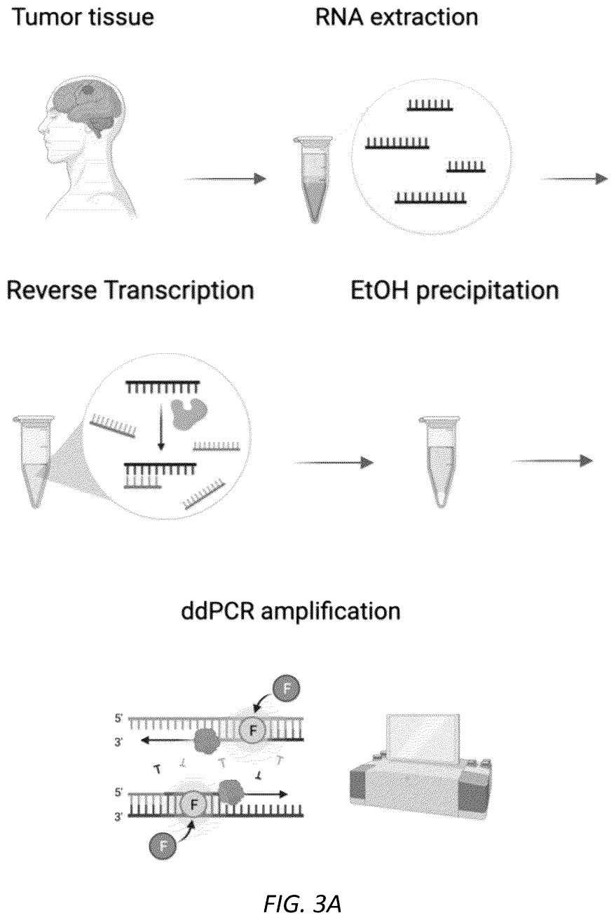

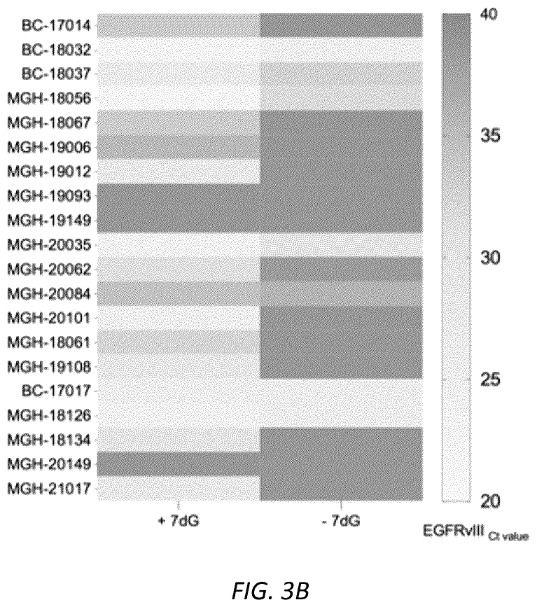

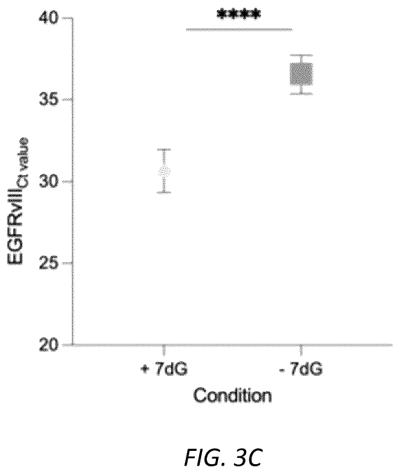

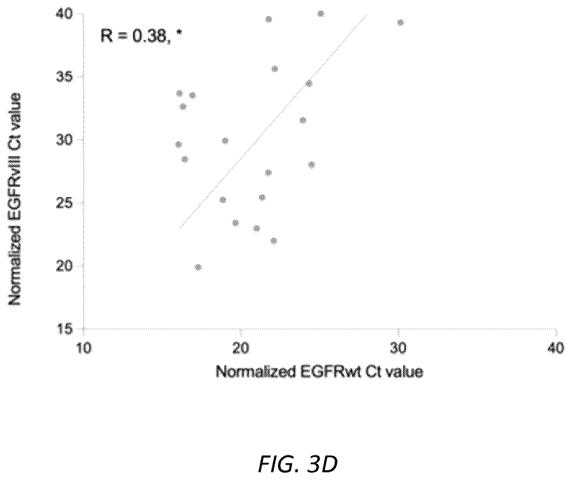

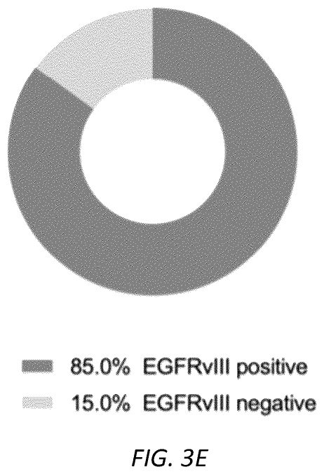

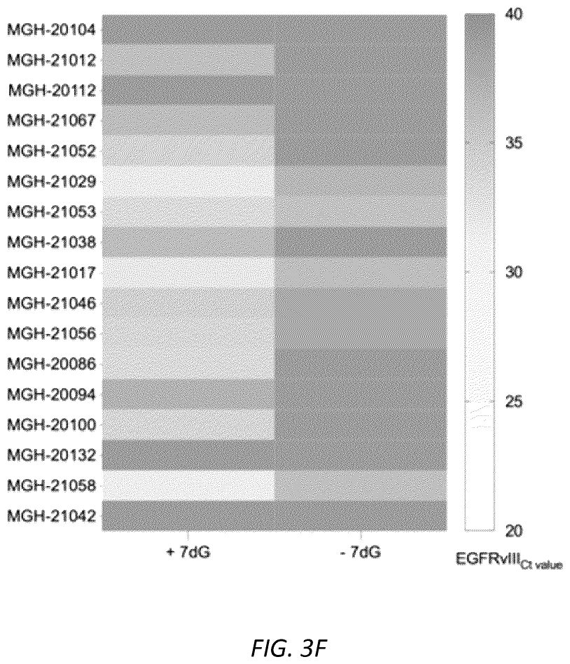

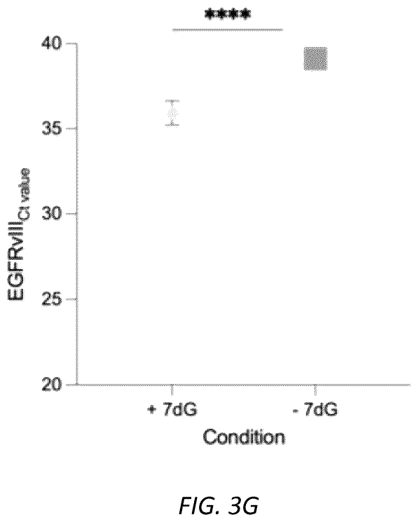

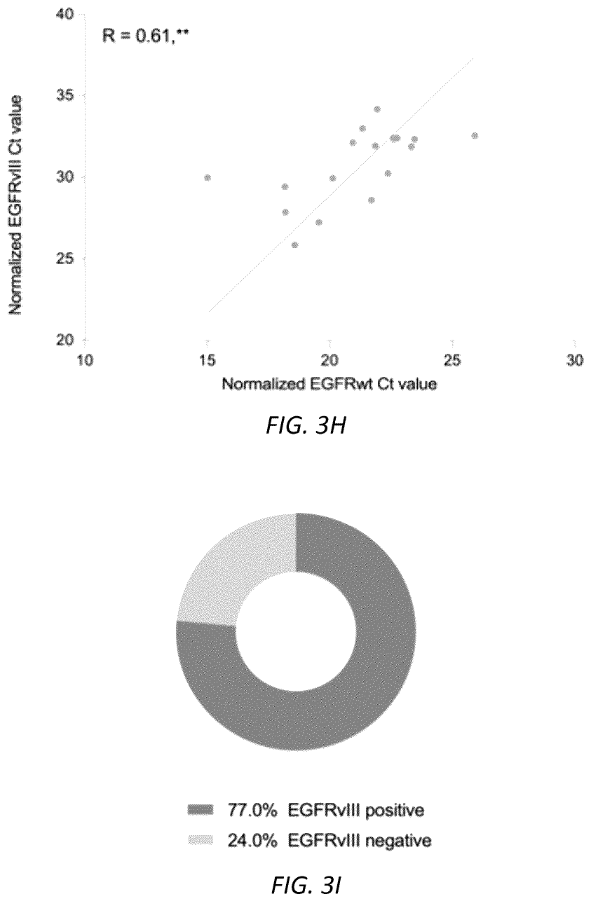

Two independent cohorts were compiled for testing. Cohort 2 included 20 patients with histopathologically confirmed GBM and EGFR amplified status. Cohort 3 included 17 patients with histopathologically confirmed GBM and EGFR amplification confirmed by qPCR. No tumor tissue was available from cohort 1 and the EGFRvIII status was histopathologically confirmed. Total RNA input for Reverse transcription (RT) of all samples was normalized to 1 ng, based on quantification data from Agilent Technologies 2100 Bioanalyzer (Waldbronn, Germany). Extracted RNA from each sample was then reverse transcribed to cDNA using two different RT conditions: with and without 7 dG (See Methods). The resulting cDNA from the two RT conditions was then amplified in two replicates per condition using the optimized qPCR protocol (FIG. 3a). Results were tabulated by comparing Ct values across both conditions (i.e., with and without 7 dG; FIG. 3 b, c, f, g).

Across all 37 tumor tissue RNA samples (cohorts 2 and 3), 7 dG treated cDNA was consistently associated with significantly lower Ct values and hence higher EGFRvIII concentration (FIG. 3 b,f respectively; (average Ct; cohort 2=30.65 vs 36.55, cohort 3=35.92 vs 39.12). This difference was statistically significant (FIG. 3 c,g;p<0.0001). The data confirmed and validated our preliminary findings that the addition of 7 dG to the RT significantly improved EGFRvIII detection. Using the assay without 7 dG resulted in a statistically significantly lower EGFRvIII detection with only 7/20 (35%) patients testing positive in cohort 2 and 6/17 (35%) in cohort 3 (FIG. 3 e,i). To confirm EGFR amplification status we normalized EGFRwt using GAPDH and performed Pearson's correlation test against EGFRvIII (also normalized to GAPDH) and confirmed a statistically significant positive correlation between EGFRvIII and EGFRwt in cohorts 2 and cohort 3 (FIG. 3 d, h, respectively).

In summary, use of 7 dG was associated with a significant improvement in tumor tissue assay sensitivity. This statistically significant finding was rigorous and reproducible in two independent tumor tissue cohorts.

### Example 4. Detection of EGFRvIII Mutation in Plasma Derived EV RNA from Three Independent Cohorts

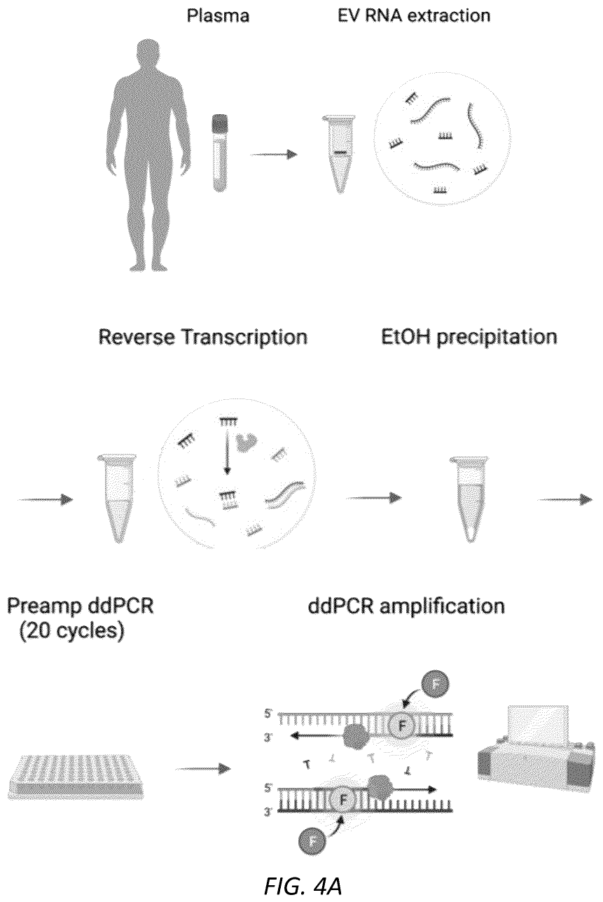

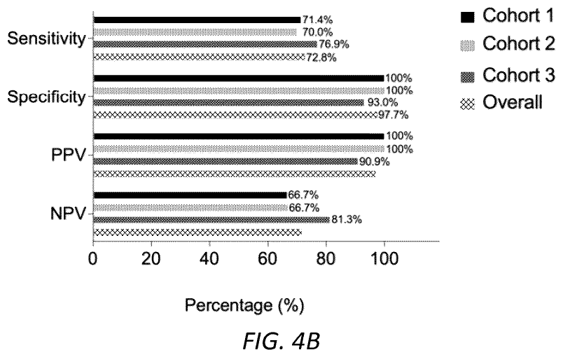

Finally, the optimized ddPCR workflow (FIG. 4a) was applied to the detection of the EGFRvIII mutation in EV RNA isolated from patient plasma. Baseline plasma collected prior to surgical resection was used for testing. For this purpose, three independent cohorts were analyzed (Table 1). Overall, our study population (n=54) comprised of n=30 histopathologically confirmed GBM (cohort 1, n=7, cohort 2, n=10, cohort 3, n=13) patients, n=10 EGFRwt controls (cohort 1 n=1, cohort 2, n=2, cohort 3, n=7), and n=14 age matched healthy controls (cohort 1, n=3, cohort 2, n=4, cohort 3, n=7). In addition, four GBM patients with longitudinal plasma available were included in cohort 3 to evaluate the clinical applicability of our assay in disease monitoring and surveillance. Plasma samples were analyzed and gated using predefined criteria (see Methods).

In cohort 1, we report positivity in 5 of 7 EGFRvIII mutant samples and none of the 4 controls (EGFR wt, healthy controls), with a sensitivity of 71.4% and specificity of 100% (FIG. 4c, Table 1). We then verified the assay performance in a second independent cohort (cohort 2) of 16 plasma samples (EGFRvIII mutant n=10, EGFR wt n=2, healthy controls n=4) (FIG. 4c, Table 1). Here we report positivity in 7 of 10 EGFRvIII mutant samples and none of the 6 controls (EGFR wt, healthy controls), with a sensitivity of 70.0% and specificity of 100% (FIG. 4c).

Lastly, the assay performance was validated in a third independent cohort, blinded to the operator (Total n=27, EGFRvIII mutant n=13, EGFRwt n=7, healthy controls n=7) (Table 1). Here we report a positivity in 10 of 13 EGFRvIII mutant samples and only 1 of the 14 controls (EGFR wt sample, false positive), with a sensitivity of 76.9% and specificity of 93% (FIG. 4c). Importantly, the sample reported as false positive had on average 1 mutant copy in the four replicates (only two out of four replicates had positive events) and the patient was confirmed to have an EGFR amplified glioblastoma. The blinded cohort using a large number of disease and non-disease controls, verifies and validates the clinically relevant sensitivity and specificity of our assay for the detection of EGFRvIII mutation in patient plasma derived EV RNA.

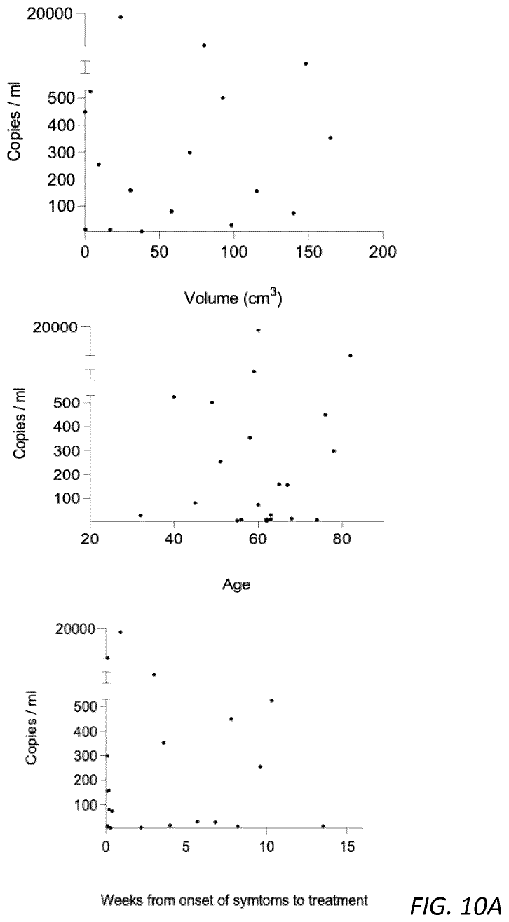

In summary, combining all three cohorts (n=54), we present an assay with overall sensitivity of 72.77% (95% CI, 63.71%-81.83%), specificity of 97.67% (CI, 87.63%-100%), positive predictive value (PPV) of 97.90%, and negative predictive value (NPV) of 70.04%. Overall, the plasma detection of EGFRvIII did not significantly correlate with different clinical parameters (FIG. 10 a,b). However, the copies/ml did show a tendency to correlation (p-value not statistically significant) with tumor volume, age, and progression free survival with higher copies likely to be detected in higher tumor volumes, older patients, and more aggressive disease.

TABLE 1
Patient Demographics

| | Cohort | | Cohort | | Cohort | |
|---|---|---|---|---|---|---|
| | 1 | % | 2 | % | 3 | % |
| Total | 11 | | 16 | | 27 | |
| Age median, | 53 | | 62 | | 64 | |
| (yr) | | | | | | |

Sex

| | | | | | | |
|---|---|---|---|---|---|---|
| Male | 8 | 72.7 | 11 | 68.8 | 16 | 59.3 |
| Female | 3 | 27.3 | 5 | 31.3 | 11 | 40.1 |

Race, n (%)

| | | | | | | |
|---|---|---|---|---|---|---|
| White | 8 | 72.7 | 12 | 75 | 19 | 70.4 |
| Asian | | | | | | |
| Other | | | | | | |
| Unavailable | 3 | 27.3 | 4 | 25 | 8 | 29.6 |

Ethnicity, n (%)

| | | | | | | |
|---|---|---|---|---|---|---|
| Hispanic | | | | | | |
| Non-Hispanic | 8 | 72.7 | 12 | 75 | 19 | 70.4 |
| Unavailable | 3 | 27.3 | 4 | 25 | 8 | 29.6 |
| WHO Grade, n | 8 | | 12 | | 20 | |
| (%) (from | | | | | | |
| diseased total) | | | | | | |
| II | | | | | | |
| III | | | 1 | 8.3 | 2 | 10 |
| IV | 7 | 87.5 | 11 | 91.7 | 17 | 85 |
| N/A | 1 | 12.5 | | | 1 | 5 |

Disease Status (from diseased total)

| | | | |
|---|---|---|---|
| Newly | 1 | 6 | 16 |
| Diagnosed | | | |
| Recurrent | 6 | 3 | 4 |
| N/A | 1 | 3 | |

Medications, n (%) (from diseased total)

| | | | | | | |
|---|---|---|---|---|---|---|
| Anticonvulsants | 5 | 62.5 | 9 | 75 | 10 | 50 |
| (levetriacetam, | | | | | | |
| clonazepam, | | | | | | |
| lacosomide, | | | | | | |
| carbamazepine, | | | | | | |
| lamotrigine) | | | | | | |
| Steroids | 7 | 87.5 | 5 | 41.7 | 4 | 20 |
| (dexamethasone) | | | | | | |
| Blood thinner | | | 3 | 25 | 5 | 25 |
| (aspirin, | | | | | | |
| coumadin) | | | | | | |
| Prior radiation | | | | | | |
| therapy | | | | | | |
| Prior | | | | | | |
| chemotherapy | | | | | | |
| Clinical trial | 3 | 37.5 | 1 | 8.3 | 1 | 5 |
| participant | | | | | | |

### Example 5. Longitudinal Monitoring Using Patient Plasma

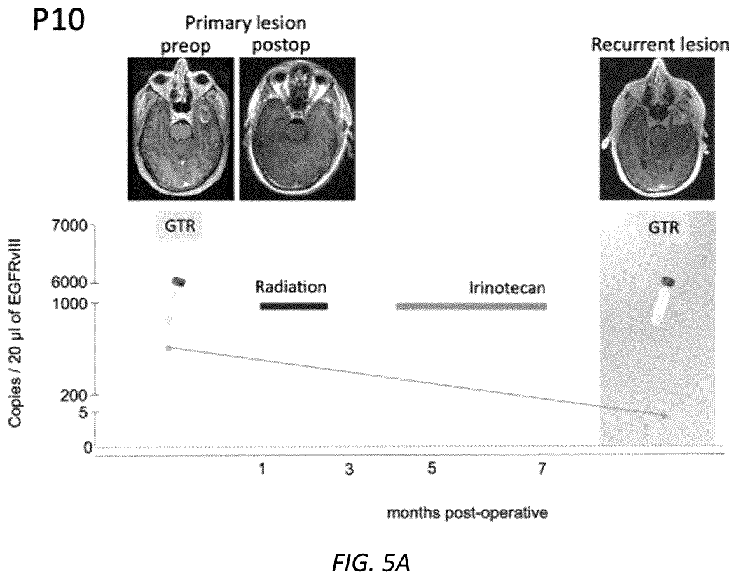

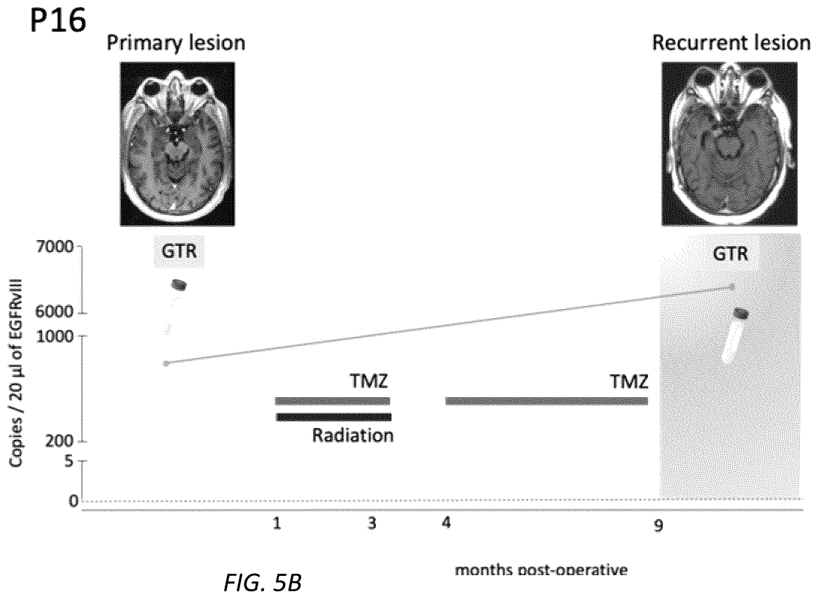

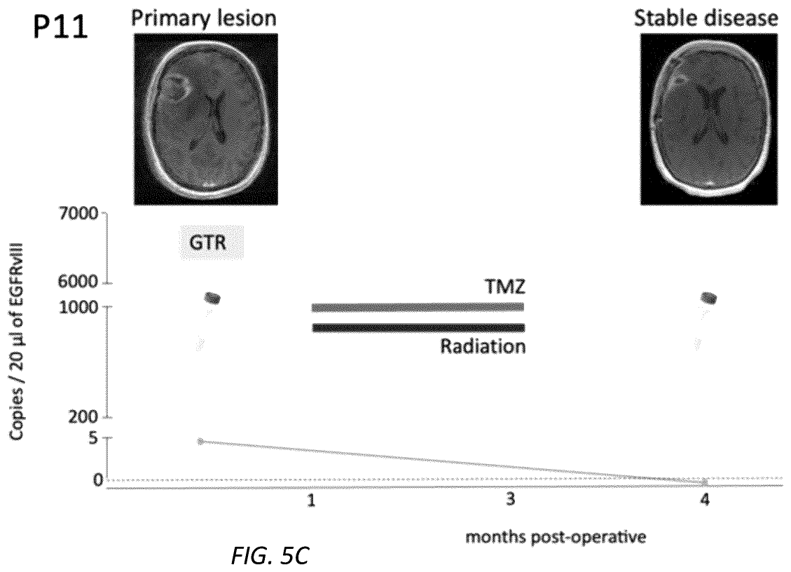

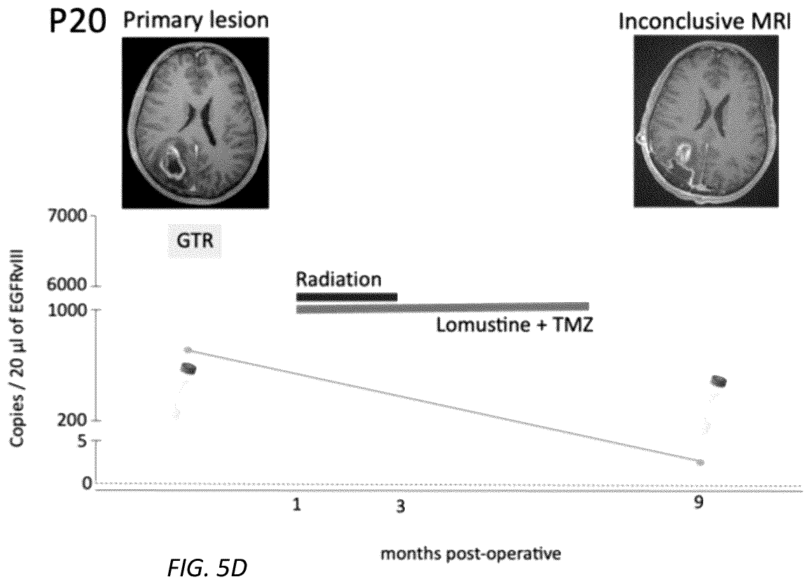

To assess the performance of EGFRvIII assay in a longitudinal setting, we isolated and analyzed EV RNA from serial plasma samples of four glioma patients, tissue confirmed to harbor the EGFRvIII mutation (FIG. 5). We also report assay reproducibility across a range of EGFRvIII and EGFRwt plasma samples analyzed multiple times (2 ml each run; FIG. 10b). EGFRvIII mutant copies correlated with the clinical and radiological findings. Each of the patients had detectable EGFRvIII copies prior to the initial surgical resection. On follow-up, P3 had stable disease following chemoradiation with no evidence of progression on radiology. Neurological examination in clinical settings was also unremarkable. This was supported by the ddPCR results with no detectable EGFRvIII mutant copies in plasma samples collected at the same time (FIG. 5c). Patients P1 and P2 developed tumor recurrence with significant progression of contrast enhancement on surveillance MRI. Furthermore, there was clinical deterioration warranting a second surgery. In both patients, plasma collected prior to second surgery demonstrated EGFRvIII mutant copies coincident with the radiological and clinical findings (FIG. 5a,b, respectively). Lastly, Patient P4 is on tumor surveillance post treatment with dual alkylating chemotherapy and radiation. The surveillance MRI demonstrates an irregular increase in contrast enhancement extending beyond the margins of the resection cavity. In other words, tumor progression cannot be ruled out with the possibility of viable tumor along the margins of the initial reaction cavity. As per clinical documentation the patient also reports occasional non-specific neurological signs. Plasma analysis of EV RNA corresponding to this time point via our ddPCR assay demonstrates detectable EGFRvIII mutant copies (FIG. 5d). Given the radiological findings, this patient will be followed to monitor clinical symptoms and progression versus stability of the MRI findings.

### Example 6. Detection of EGFRvIII Mutation in CSF and Plasma Derived EV RNA

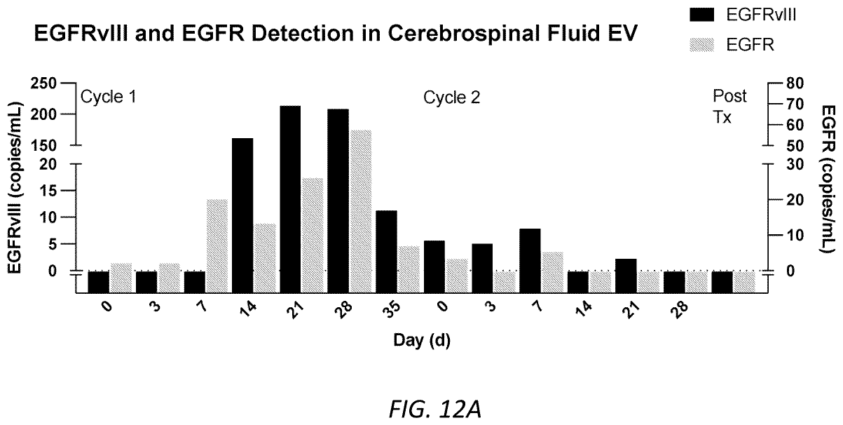

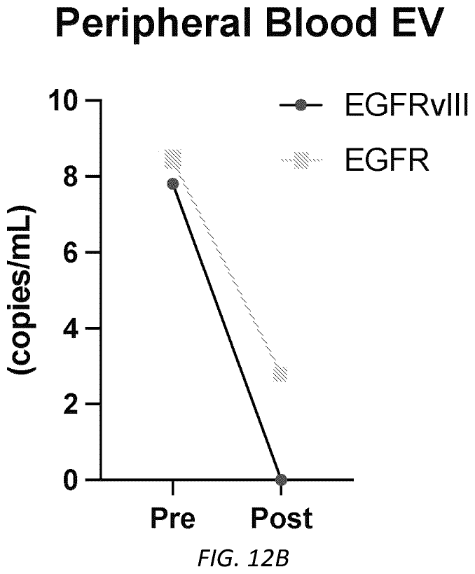

Liquid biopsy was performed on extracellular vesicles (EV) derived from cerebrospinal fluid (CSF) and peripheral blood from a human patient diagnosed with GBM using the methods described above at several time points throughout two treatment cycles with a targeted immunotherapy. EGFRvIII and EGFR copy numbers were elevated during a first treatment cycle and decreased over time, eventually becoming undetectable in post-treatment CSF (FIG. 12A). Peripheral blood samples from pre- and post-study timepoints similarly demonstrated decrease in copy number of both EGFRvIII and EGFR (FIG. 12B). These results demonstrated that CSF can also be used in the present methods.

### REFERENCES

1. Agnihotri, S. et al. Glioblastoma, a brief review of history, molecular genetics, animal models and novel therapeutic strategies. Arch. Immunol. Ther. Exp. 61, 25-41 (2013).
2. Messali, A., Villacorta, R. & Hay, J. W. A review of the economic burden of glioblastoma and the cost effectiveness of pharmacologic treatments.

Pharmacoeconomics 32, 1201-1212 (2014).
3. Taylor, O. G., Brzozowski, J. S. & Skelding, K. A. Glioblastoma Multiforme: An Overview of Emerging Therapeutic Targets. Front. Oncol. 9, 963 (2019).
4. Ostrom, Q. T. et al. CBTRUS Statistical Report: Primary Brain and Central Nervous System Tumors Diagnosed in the United States in 2008-2012.

Neuro. Oncol. 17 Suppl 4, iv1-iv62 (2015).
5. Baid, U. et al. Overall Survival Prediction in Glioblastoma With Radiomic Features Using Machine Learning. Front. Comput. Neurosci. 14, 61 (2020).
6. Saenz-Antoñanzas, A. et al. Liquid Biopsy in Glioblastoma: Opportunities, Applications and Challenges. Cancers 11, (2019).
7. Santiago-Dieppa, D. R. et al. Extracellular vesicles as a platform for ‘liquid biopsy’ in glioblastoma patients. Expert Rev. Mol. Diagn. 14, 819-825 (2014).
8. Garcia-Romero, N. et al. DNA sequences within glioma-derived extracellular vesicles can cross the intact blood-brain barrier and be detected in peripheral blood of patients. Oncotarget 8, 1416-1428 (2017).
9. Skog, J. et al. Glioblastoma microvesicles transport RNA and proteins that promote tumour growth and provide diagnostic biomarkers. Nat. Cell Biol. 10, 1470-1476 (2008).
10. Figueroa, J. M. et al. Detection of wild-type EGFR amplification and EGFRvIII mutation in CSF-derived extracellular vesicles of glioblastoma patients. Neuro. Oncol. 19, 1494-1502 (2017).
11. Manda, S. V. et al. Exosomes as a biomarker platform for detecting epidermal growth factor receptor-positive high-grade gliomas. J. Neurosurg. 128, 1091-1101 (2018).
12. Oprita, A. et al. Updated Insights on EGFR Signaling Pathways in Glioma. Int. J. Mol. Sci. 22, (2021).
13. An, Z., Aksoy, O., Zheng, T., Fan, Q.-W. & Weiss, W. A. Epidermal growth factor receptor and EGFRvIII in glioblastoma: signaling pathways and targeted therapies. Oncogene 37, 1561-1575 (2018).
14. The EGFRvIII variant in glioblastoma multiforme. J. Clin. Neurosci. 16, 748-754 (2009).
15. Kalman, B., Szep, E., Garzuly, F. & Post, D. E. Epidermal growth factor receptor as a therapeutic target in glioblastoma. Neuromolecular Med. 15, 420-434 (2013).
16. Varshney, D., Spiegel, J., Zyner, K., Tannahill, D. & Balasubramanian, S. The regulation and functions of DNA and RNA G-quadruplexes. Nat. Rev. Mol. Cell Biol. 21, 459-474 (2020).
17. Brierley, I., Pennell, S. & Gilbert, R. J. C. Viral RNA pseudoknots: versatile motifs in gene expression and replication. Nat. Rev. Microbiol. 5, 598-610 (2007).
18. Fan, H., Wang, J., Komiyama, M. & Liang, X. Effects of secondary structures of DNA templates on the quantification of qPCR. J. Biomol. Struct. Dyn. 37, 2867-2874 (2019).
19. Lyubetsky, V. A., Rubanov, L. I., Seliverstov, A. V. & Pirogov, S. A. Model of gene expression regulation in bacteria via formation of RNA secondary structures. Mol. Biol. 40, 440-453 (2006).
20. Muralidharan, K. et al. Promoter Mutation Analysis for Blood-Based Diagnosis and Monitoring of Gliomas. Clin. Cancer Res. 27, 169-178 (2021).
21. Zuker, M. & Stiegler, P. Optimal computer folding of large RNA sequences using thermodynamics and auxiliary information. Nucleic Acids Res. 9, 133-148 (1981).
22. Zuker, M. Mfold web server for nucleic acid folding and hybridization prediction. Nucleic Acids Res. 31, 3406-3415 (2003).
23. Nussinov, R., Pieczenik, G., Griggs, J. R. & Kleitman, D. J. Algorithms for Loop Matchings. SIAM J. Appl. Math. 35, 68-82 (1978).
24. Frey, U. H., Bachmann, H. S., Peters, J. & Siffert, W. PCR-amplification of GC-rich regions: ‘slowdown PCR’. Nat. Protoc. 3, 1312-1317 (2008).
25. Corless, B. C. et al. Development of Novel Mutation-Specific Droplet Digital PCR Assays Detecting TERT Promoter Mutations in Tumor and Plasma Samples. J. Mol. Diagn. 21, 274-285 (2019).
26. Sun, K. et al. Orientation-aware plasma cell-free DNA fragmentation analysis in open chromatin regions informs tissue of origin. Genome Res. 29, 418-427 (2019).
27. Musso, M., Bocciardi, R., Parodi, S., Ravazzolo, R. & Ceccherini, I. Betaine, dimethyl sulfoxide, and 7-deaza-dGTP, a powerful mixture for amplification of GC-rich DNA sequences. J. Mol. Diagn. 8, 544-550 (2006).
28. Rees, W. A., Yager, T. D., Korte, J. & von Hippel, P. H. Betaine can eliminate the base pair composition dependence of DNA melting. Biochemistry 32, 137-144 (1993).
29. Heimberger, A. B. et al. Prognostic effect of epidermal growth factor receptor and EGFRvIII in glioblastoma multiforme patients. Clin. Cancer Res. 11, 1462-1466 (2005).
30. Felsberg, J. et al. Epidermal Growth Factor Receptor Variant III (EGFRvIII) Positivity in-Amplified Glioblastomas: Prognostic Role and Comparison between Primary and Recurrent Tumors. Clin. Cancer Res. 23, 6846-6855 (2017).
31. Sugawa, N., Ekstrand, A. J., James, C. D. & Collins, V. P. Identical splicing of aberrant epidermal growth factor receptor transcripts from amplified rearranged genes in human glioblastomas. Proc. Natl. Acad. Sci. U.S.A 87, 8602-8606 (1990).
32. Ekstrand, A. J., Sugawa, N., James, C. D. & Collins, V. P. Amplified and rearranged epidermal growth factor receptor genes in human glioblastomas reveal deletions of sequences encoding portions of the N- and/or C-terminal tails. Proceedings of the National Academy of Sciences vol. 89 4309-4313 (1992).
33. Rutkowska, A., Stoczynska-Fidelus, E., Janik, K., Wlodarczyk, A. & Rieske, P. EGFRvIII: An Oncogene with Ambiguous Role. J. Oncol. 2019, (2019).
34. Wong, A. J. et al. Structural alterations of the epidermal growth factor receptor gene in human gliomas. Proc. Natl. Acad. Sci. U.S.A 89, 2965-2969 (1992).
35. Del Vecchio, C. A. et al. EGFRvIII gene rearrangement is an early event in glioblastoma tumorigenesis and expression defines a hierarchy modulated by epigenetic mechanisms. Oncogene 32, 2670-2681 (2013).
36. Nozawa, T. et al. EGFRvIII Is Expressed in Cellular Areas of Tumor in a Subset of Glioblastoma. Neurol. Med. Chir. 59, 89-97 (2019).
37. Batool, S. M. et al. Highly Sensitive EGFRvIII Detection in Circulating Extracellular Vesicle RNA of Glioma Patients. Clin. Cancer Res. 28, 4070-4082 (2022).
38. Gan, H. K., Cvrljevic, A. N. & Johns, T. G. The epidermal growth factor receptor variant III (EGFRvIII): where wild things are altered. FEBS J. 280, 5350-5370 (2013).
39. Ge, H., Gong, X. & Tang, C. K. Evidence of high incidence of EGFRvIII expression and coexpression with EGFR in human invasive breast cancer by laser capture microdissection and immunohistochemical analysis. Int. J. Cancer 98, 357-361 (2002).
40. Sonnweber, B. et al. High predictive value of epidermal growth factor receptor phosphorylation but not of EGFRvIII mutation in resected stage I non-small cell lung cancer (NSCLC). J. Clin. Pathol. 59, 255-259 (2006).
41. Garcia de Palazzo, I. E. et al. Expression of mutated epidermal growth factor receptor by non-small cell lung carcinomas. Cancer Res. 53, 3217-3220 (1993).
42. Okamoto, I. et al. Expression of constitutively activated EGFRvIII in non-small cell lung cancer. Cancer Sci. 94, 50-56 (2003).
43. Sok, J. C. et al. Mutant epidermal growth factor receptor (EGFRvIII) contributes to head and neck cancer growth and resistance to EGFR targeting. Clin.

Cancer Res. 12, 5064-5073 (2006).
44. Thomas Koch, D. et al. Epidermal growth factor receptor variant III in head and neck squamous cell carcinoma is not relevant for targeted therapy and irradiation. Oncotarget 8, 32668-32682 (2017).
45. Peciak, J. et al. Low Incidence along with Low mRNA Levels of EGFRvIII in Prostate and Colorectal Cancers Compared to Glioblastoma. J. Cancer 8, 146-151 (2017).

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method comprising:
providing a sample comprising a serum or cerebrospinal fluid from a subject, preferably a human subject;
isolating extracellular vesicles comprising RNA from the sample;
reverse transcribing the RNA in the presence of 7-deaza-deoxyguanosine 5′-triphosphate (7 dG) to create cDNA;
using polymerase chain reaction (PCR), amplifying any epidermal growth factor receptor v III (EGFRvIII) sequences present in the cDNA to generate EGFRvIII amplicons, preferably wherein the amplicons are between 50 and 100 bp in length;
detecting the presence of EGFRvIII amplicons.

2. The method of claim 1, wherein the method includes a step of pre-amplifying the EGFRvIII sequences.

3. The method of claim 1, wherein the sample is from a subject who is known or suspected to have cancer.

4. The method of claim 3, wherein the cancer is glioma, breast cancer, non-small cell lung cancer, head & neck squamous cell carcinoma, colorectal cancer, or prostate cancer.

5. The method of claim 4, wherein the glioma is glioblastoma.

6. The method of claim 1, wherein the amplification is performed using Forward primer (5′-GGCTCTGGAGGAAAAGAAAGGTAATT-3′; SEQ ID NO: 8) and/or Reverse primer (5′-CCGTCTTCCTCCATCTCATAGC-3′; SEQ ID NO: 9).

7. The method of claim 2, wherein the preamplification is performed using Forward primer (5′-GGCTCTGGAGGAAAAGAAAGGTAATT-3′; SEQ ID NO:8) and/or Reverse primer (5′-CCGTCTTCCTCCATCTCATAGC-3′; SEQ ID NO: 9).

8. The method of claim 1, wherein the amplification is performed using digital droplet PCR, preferably in the presence of betaine.

9. The method of claim 2, wherein the pre-amplification is performed using digital droplet PCR, preferably in the presence of betaine.

10. The method of claim 1, wherein the amplification is performed in the presence of an EGFRvIII mutant probe comprising a detectable moiety, and detecting the presence of EGFRvIII amplicons comprises detecting amplicons comprising the detectable moiety.

11. The method of claim 10, wherein the probe comprises 5′-TGACAGATCACGGCTC-3′; SEQ ID NO: 10.

12. A method of detecting an EGFRvIII positive cancer in a human subject who is known or suspected to have a cancer, the method comprising:
providing a sample comprising a serum or cerebrospinal fluid from the subject;
isolating extracellular vesicles comprising RNA from the sample;
reverse transcribing the RNA in the presence of 7-deaza-deoxyguanosine 5′-triphosphate (7 dG) to create cDNA;
using polymerase chain reaction (PCR), amplifying any epidermal growth factor receptor v III (EGFRvIII) sequences present in the cDNA to generate EGFRvIII amplicons, preferably wherein the amplicons are between 50 and 100 bp in length;
detecting the presence of EGFRvIII amplicons, wherein the presence of EGFRvIII amplicons indicates that the cancer is EGFRvIII positive.

13. The method of claim 12, further comprising administering to the subject a treatment for EGFRvIII positive cancer, optionally EGFR inhibitors;
immunotherapy; erb-B inhibitors; VEGF/VEGFR inhibitors, surgical resection, and radiation therapy, as well as combinations thereof.

14. The method of claim 12, wherein the method includes a step of pre-amplifying the EGFRvIII sequences.

15. The method of claim 14, wherein the cancer is glioma, breast cancer, non-small cell lung cancer, head & neck squamous cell carcinoma, colorectal cancer, or prostate cancer.

16. The method of claim 15, wherein the glioma is glioblastoma.

17. The method of claim 12, wherein the amplification is performed using Forward primer (5′-GGCTCTGGAGGAAAAGAAAGGTAATT-3′; SEQ ID NO: 8) and/or Reverse primer (5′-CCGTCTTCCTCCATCTCATAGC-3′; SEQ ID NO: 9).

18. The method of claim 14, wherein the preamplification is performed using Forward primer (5′-GGCTCTGGAGGAAAAGAAAGGTAATT-3′; SEQ ID NO:8) and/or Reverse primer (5′-CCGTCTTCCTCCATCTCATAGC-3′; SEQ ID NO: 9).

19. The method of claim 12, wherein the amplification is performed using digital droplet PCR, preferably in the presence of betaine.

20. The method of claim 14, wherein the pre-amplification is performed using digital droplet PCR, preferably in the presence of betaine.

21. The method of claim 12, wherein the amplification is performed in the presence of an EGFRvIII mutant probe comprising a detectable moiety, and detecting the presence of EGFRvIII amplicons comprises detecting amplicons comprising the detectable moiety, optionally wherein the probe comprises 5′-TGACAGATCACGGCTC-3′; SEQ ID NO: 10.


SEQUENCE LISTING (34 sequences)

SEQ ID NO: 1 - DNA, 96 bp, synthetic construct
  source 1..96: mol_type = other DNA
ggctctggaggaaaagaaaggtaattatgtggtgacagatcacggctcgtgcgtccgagcctgtggggccgacagctatgagatggaggaagacgg

SEQ ID NO: 2 - DNA, 62 bp, synthetic construct
  source 1..62: mol_type = other DNA
tatgtcctcattgccctcaacacagtggagcgaattcctttggaaaacctgcagatcatcag

SEQ ID NO: 3 - RNA, 109 nt, synthetic construct
  source 1..109: mol_type = other RNA
cgagtcgggctctggaggaaaagaaaggtaattatgtggtgacagatcacggctcgtgcgtccgagcctgtggggccgacagctatgagatggaggaagacggcgtccg

SEQ ID NO: 4 - RNA, 62 nt, synthetic construct
  source 1..62: mol_type = other RNA
tatgtcctcattgccctcaacacagtggagcgaattcctttggaaaacctgcagatcatcag

SEQ ID NO: 5 - RNA, 120 nt, synthetic construct
  source 1..120: mol_type = other RNA
gcccggcgagtcgggctctggaggaaaagaaaggtaattatgtggtgacagatcacggctcgtgcgtccgagcctgtggggccgacagctatgagatggaggaagacggcgtccgcaagt

SEQ ID NO: 6 - DNA, 62 bp, synthetic construct; identical to SEQ ID NO: 2
  source 1..62: mol_type = other DNA

SEQ ID NO: 7 - DNA, 466 bp, synthetic construct
  source 1..466: mol_type = other DNA
agacgtccgggcagcccccggcgcagcgcggccgcagcagcctccgccccccgcacggtgtgagcgcccgacgcggccgaggcggccggagtcccgagctagccccggcggccgccgccgcccagaccggacgacaggccacctcgtcggcgtccgcccgagtccccgcctcgccgccaacgccacaaccaccgcgcacggccccctgactccgtccagtattgatcgggagagccggagcgagctcttcggggagcagcgatgcgaccctccgggacggccggggcagcgctcctggcgctgctggctgcgctctgcccggcgagtcgggctctggaggaaaagaaaggtaattatgtggtgacagatcacggctcgtgcgtccgagcctgtggggccgacagctatgagatggaggaagacggcgtccgcaagtgtaagaagtgcgaagggccttgccgcaaag

SEQ ID NO: 8 - DNA, 26 bp, synthetic construct
  source 1..26: mol_type = other DNA
ggctctggaggaaaagaaaggtaatt

SEQ ID NO: 9 - DNA, 22 bp, synthetic construct
  source 1..22: mol_type = other DNA
ccgtcttcctccatctcatagc

SEQ ID NO: 10 - DNA, 16 bp, synthetic construct
  source 1..16: mol_type = other DNA
tgacagatcacggctc

SEQ ID NO: 11 - DNA, 17 bp, synthetic construct
  source 1..17: mol_type = other DNA
ggacgacaggccacctc

SEQ ID NO: 12 - DNA, 20 bp, synthetic construct
  source 1..20: mol_type = other DNA
ttcttttcctccagagcccg

SEQ ID NO: 13 - DNA, 17 bp, synthetic construct
  source 1..17: mol_type = other DNA
ggacgacaggccacctc

SEQ ID NO: 14 - DNA, 20 bp, synthetic construct
  source 1..20: mol_type = other DNA
ccgcaagtgtaagaagtgcg

SEQ ID NO: 15 - DNA, 17 bp, synthetic construct
  source 1..17: mol_type = other DNA
ggacgacaggccacctc

SEQ ID NO: 16 - DNA, 19 bp, synthetic construct
  source 1..19: mol_type = other DNA
ccatcagtggcgatctcca

SEQ ID NO: 17 - DNA, 16 bp, synthetic construct
  source 1..16: mol_type = other DNA
ctcctggcgctgctgg

SEQ ID NO: 18 - DNA, 20 bp, synthetic construct
  source 1..20: mol_type = other DNA
gagccgtgatctgtcaccac

SEQ ID NO: 19 - DNA, 16 bp, synthetic construct
  source 1..16: mol_type = other DNA
cggcgagtcgggctct

SEQ ID NO: 20 - DNA, 20 bp, synthetic construct
  source 1..20: mol_type = other DNA
catctcatagctgtcggccc

SEQ ID NO: 21 - DNA, 16 bp, synthetic construct
  source 1..16: mol_type = other DNA
ctggctgcgctctgcc

SEQ ID NO: 22 - DNA, 19 bp, synthetic construct
  source 1..19: mol_type = other DNA
ggacgcacgagccgtgatc

SEQ ID NO: 23 - DNA, 22 bp, synthetic construct
  source 1..22: mol_type = other DNA
gagtcgggctctggaggaaaag

SEQ ID NO: 24 - DNA, 18 bp, synthetic construct
  source 1..18: mol_type = other DNA
ccacaggctcggacgcac

SEQ ID NO: 25 - DNA, 17 bp, synthetic construct
  source 1..17: mol_type = other DNA
tcgggctctggaggaaa

SEQ ID NO: 26 - DNA, 22 bp, synthetic construct
  source 1..22: mol_type = other DNA
cttcctccatctcatagctgtc

SEQ ID NO: 27 - DNA, 22 bp, synthetic construct
  source 1..22: mol_type = other DNA
cttcggggagcagcgatgcgac

SEQ ID NO: 28 - DNA, 22 bp, synthetic construct
  source 1..22: mol_type = other DNA
accaatacctattccgttacac

SEQ ID NO: 29 - DNA, 20 bp, synthetic construct
  source 1..20: mol_type = other DNA
atgcgaccctccgggacggc

SEQ ID NO: 30 - DNA, 22 bp, synthetic construct
  source 1..22: mol_type = other DNA
accaatacctattccgttacac

SEQ ID NO: 31 - DNA, 18 bp, synthetic construct
  source 1..18: mol_type = other DNA
atgcgaccctccgggacg

SEQ ID NO: 32 - DNA, 18 bp, synthetic construct
  source 1..18: mol_type = other DNA
gagtatgtgtgaaggagt

SEQ ID NO: 33 - DNA, 20 bp, synthetic construct
  source 1..20: mol_type = other DNA
ccagtattgatcgggagagc

SEQ ID NO: 34 - DNA, 22 bp, synthetic construct
  source 1..22: mol_type = other DNA
tcagaatatccagttcctgtgg